(19) United States
(12) Patent Application Publication
(10) Pub. No.: US 2026/0294761 A1
(43) Pub. Date: Oct. 1, 2026

(54) SULFATE-FREE AND PEG-FREE FOAMING CLEANSING COMPOSITIONWITH STABILIZED CATIONIC CONDITIONING POLYMERS POLYQUATERNIUM-22, 39, 47, 67 AND NATURAL THICKENER LAURYL LACTATE

(71) Applicant: L'OREAL, Paris (FR)

(72) Inventors: Hungta LIN, Teaneck, NJ (US); Christopher Pang, New York, NY (US); Changlong Chen, Edison, NJ (US); Mariko Hasebe, New York, NY (US)

(73) Assignee: L'OREAL, Paris (FR)

(21) Appl. No.: 19/095,852

(22) Filed: Mar. 31, 2025

(51) Int. Cl. A61K 8/37; A61K 8/02; A61K 8/04; A61K 8/44; A61K 8/81; A61Q 5/02; A61Q 19/10

(52) U.S. Cl. CPC A61K 8/37; A61K 8/0291; A61K 8/042; A61K 8/447; A61K 8/817; A61Q 5/02; A61Q 19/10; A61K 2800/33; A61K 2800/48

(57) ABSTRACT

The invention relates to a micellar aqueous cleansing composition featuring a natural thickener comprising lauryl lactate and at least one or a combination of polyquaternium cationic polymers. This composition is formulated to exclude any added PEG-based ingredients, including PEG/PEO polymeric thickeners, as well as sulfate-based ingredients, such as sulfate-based surfactants. The exclusion of these ingredients is aimed at reducing potential irritants and synthetic compounds. The resulting formulation achieves a gel-like viscosity and maintains a transparent appearance, offering a gentle and effective cleansing solution with improved stability and skin compatibility.

### FIELD

The present invention relates to a micellar aqueous cleansing composition, specifically formulation embodiments that utilize natural thickener comprising lauryl lactate and polyquaternium cationic polymers, while being free from PEG-based and sulfate-based ingredients, suitable for personal care applications such as skin and hair cleansing products.

### BACKGROUND

Conventional cleansing formulations often rely on PEG-based thickeners and sulfate-based surfactants to achieve desired viscosity and cleansing efficacy. However, these ingredients can lead to concerns regarding skin irritation and environmental impact. PEG-based thickeners, for instance, are synthetic and may cause adverse reactions in sensitive skin. Similarly, sulfate-based surfactants, while effective in creating foam and removing dirt, can strip the skin of natural oils, leading to dryness and irritation.

The beauty and personal care industry is increasingly shifting towards formulations that minimize synthetic components in favor of natural and less harsh alternatives. This shift is driven by consumer demand for products that are not only effective but also gentle on the skin and environmentally friendly. Despite the availability of natural ingredients, achieving the desired cleansing performance and product stability without synthetic thickeners and surfactants remains a challenge.

The present invention addresses these challenges by formulating micellar aqueous cleansing composition s that incorporate natural thickeners and polyquaternium cationic polymers to achieve stability, viscosity, and transparency, while excluding PEG-based and sulfate-based ingredients, thus providing a more skin-friendly and eco-friendly alternative.

### SUMMARY

This disclosure describes exemplary embodiments in accordance with the general inventive concepts and is not intended to limit the scope of the invention in any way. Indeed, the invention as described in the specification is broader than and unlimited by the exemplary embodiments set forth herein, and the terms used herein have their full ordinary meaning.

The disclosure provides a micellar aqueous cleansing composition is provided, comprising at least one natural thickener comprising lauryl lactate and at least one or a combination of polyquaternium cationic polymers. The composition excludes any added polyethylene glycol (PEG), also known as polyethylene oxide (PEO), based ingredients (PEG/PEO based ingredients), including PEG/PEO polymeric thickeners, and excludes sulfate-based ingredients, including sulfate-based surfactants. The micellar aqueous cleansing composition has a gel-like viscosity and is transparent in appearance, offering a stable micellar phase free of separation at ambient and high temperatures. The micellar aqueous cleansing composition may also include amphoteric surfactants such as hydroxysultaine and anionic surfactants like sodium methyl cocoyl taurate, which contribute to the cleansing efficacy and skin compatibility of the product. Additional ingredients can include hexylene glycol, sodium hydroxide, menthol, zinc gluconate, tetrasodium glutamate diacetate, sodium benzoate, glycerin, sodium chloride, and capryloyl salicylic acid. This composition provides a gentle yet effective cleansing solution that supports skin health while aligning with consumer preferences for natural and environmentally conscious products.

According to an embodiment of the invention, the micellar aqueous cleansing composition comprises lauryl lactate present from about 0.05% to about 2%.

According to an embodiment of the invention, the micellar aqueous cleansing composition comprises lauryl lactate present from about 0.1% to about 1%.

According to an embodiment of the invention, the micellar aqueous cleansing composition comprises at least one natural thickener consisting essentially of lauryl lactate and excludes any added natural thickener. According to an embodiment of the invention, the micellar aqueous cleansing composition excludes other natural thickeners such as xanthan gum, sclerotium gum, alginate, hydroxyethylcellulose, Zea Mays starch, carrageenan.

According to an embodiment of the invention, the micellar aqueous cleansing composition comprises at least one or a combination of polyquaternium cationic polymers selected from the group consisting of polyquaternium-22, 39, 47, 67, and combinations thereof. According to an embodiment of the invention, the micellar aqueous cleansing composition comprises polyquaternium cationic polymers consisting of one of polyquaternium-22, 39, 47, or 67. According to an embodiment of the invention, the micellar aqueous cleansing composition comprises each one of the polyquaternium cationic polymers present from about at least 0.5%. According to an embodiment of the invention, the micellar aqueous cleansing composition comprises each one of the polyquaternium cationic polymers present from about 0.05% to about 1%. According to an embodiment of the invention, the micellar aqueous cleansing composition demonstrates stability including remaining visibly clear, maintaining viscosity, micellar phase stability free of separation, all at ambient temperatures and at high temperatures.

According to an embodiment of the invention, the micellar aqueous cleansing composition comprises one or more surfactants. According to an embodiment of the invention, the micellar aqueous cleansing composition comprises one or more amphoteric surfactants comprising at least hydroxysultaine and one or more anionic surfactants comprising sodium methyl cocoyl taurate. According to an embodiment of the invention, the micellar aqueous cleansing composition comprises cocoamidopropyl hydroxysultaine present in a range from about 1% to about 20%. According to an embodiment of the invention, the micellar aqueous cleansing composition comprises sodium methyl cocoyl taurate present from about 0.1% to about 1.5%.

According to an embodiment of the invention, the micellar aqueous cleansing composition comprises water present in a range from about 40% to about 90%, by weight, based on the weight of the composition.

According to an embodiment of the invention, the micellar aqueous cleansing composition comprises one or more ingredients selected from the group consisting of hexylene glycol, sodium hydroxide, menthol, zinc gluconate, tetrasodium glutamate diacetate, sodium benzoate, glycerin, sodium chloride, salicylic acid, capryloyl salicylic acid, and combinations thereof. According to an embodiment of the invention, the micellar aqueous cleansing composition comprises each of hexylene glycol, sodium hydroxide, menthol, zinc gluconate, tetrasodium glutamate diacetate, sodium benzoate, glycerin, sodium chloride, salicylic acid, capryloyl salicylic acid, and water.

According to an embodiment of the invention, the micellar aqueous cleansing composition excludes PEG-150 pentaerythrityl tetrastearate, PEG-6 caprylic/capric glycerides, or a combination thereof.

According to an embodiment of the invention, a micellar aqueous cleansing composition comprising at least one natural thickener comprising lauryl lactate, wherein lauryl lactate is present from about 0.1% to about 1%; at least one or a combination of polyquaternium cationic polymers selected from the group consisting of polyquaternium-22, 39, 47, 67, and combinations thereof, wherein each of the polyquaternium cationic polymers is present from about at least 0.5%; wherein the micellar aqueous cleansing composition excludes any added PEG-based ingredients, including PEG/PEO polymeric thickeners, and excludes sulfate-based ingredients, including sulfate-based surfactants; and wherein the micellar aqueous cleansing composition demonstrates stability including remaining visibly clear, maintaining viscosity, micellar phase stability free of separation, all at ambient temperatures and at high temperatures, has a gel-like viscosity, and is transparent in appearance.

According to an embodiment of the invention, a micellar aqueous cleansing composition comprising at least one natural thickener consisting essentially of lauryl lactate, wherein lauryl lactate is present from about 0.1% to about 1%; polyquaternium cationic polymers consisting of one of polyquaternium-22, 39, 47, or 67, wherein each of the polyquaternium cationic polymers is present from about at least 0.5%; one or more amphoteric surfactants comprising at least hydroxysultaine and one or more anionic surfactants comprising sodium methyl cocoyl taurate, wherein cocoamidopropyl hydroxysultaine is present in a range from about 1% to about 20% and sodium methyl cocoyl taurate is present from about 0.1% to about 1.5%; comprising one or more ingredients selected from the group consisting of hexylene glycol, sodium hydroxide, menthol, zinc gluconate, tetrasodium glutamate diacetate, sodium benzoate, glycerin, sodium chloride, salicylic acid, capryloyl salicylic acid, and combinations thereof; wherein the micellar aqueous cleansing composition excludes PEG-150 pentaerythrityl tetrastearate, PEG-6 caprylic/capric glycerides, or a combination thereof; and wherein the micellar aqueous cleansing composition has a gel-like viscosity and is transparent in appearance.

This micellar aqueous cleansing composition provides a gentle cleansing solution free from harsh sulfates and PEG-based thickeners, ensuring skin-friendly properties that align with consumer preferences for cleaner formulations, reducing concerns over synthetic additives, simplifying the ingredient list and enhancing consumer trust. This exclusion results in a composition that remains lightweight and transparent, offering consumers a product free from commonly used thickeners that may affect texture and appearance. The micellar aqueous cleansing composition demonstrates a clear and gel-like consistency and a visually appealing product. The micellar aqueous cleansing composition stability ensures a consistent consumer experience and product performance across various storage conditions, and the high water content ensures ease of application and rinsing, contributing to a refreshing cleansing experience.

These and other aspects of the invention are set out in the appended claims, and described in greater detail in the detailed description of the invention.

It is to be understood that both the foregoing general description and the following detailed description are exemplary and explanatory only and are not restrictive of the disclosure.

### DETAILED DESCRIPTION

The disclosure provides a micellar aqueous cleansing composition is provided, comprising at least one natural thickener comprising lauryl lactate and at least one or a combination of polyquaternium cationic polymers. The composition excludes any added polyethylene glycol (PEG), also known as polyethylene oxide (PEO), based ingredients (PEG/PEO based ingredients), including PEG/PEO polymeric thickeners, and excludes sulfate-based ingredients, including sulfate-based surfactants. The micellar aqueous cleansing composition has a gel-like viscosity and is transparent in appearance, offering a stable micellar phase free of separation at ambient and high temperatures. The micellar aqueous cleansing composition may also include amphoteric surfactants such as hydroxysultaine and anionic surfactants like sodium methyl cocoyl taurate, which contribute to the cleansing efficacy and skin compatibility of the product. Additional ingredients can include hexylene glycol, sodium hydroxide, menthol, zinc gluconate, tetrasodium glutamate diacetate, sodium benzoate, glycerin, sodium chloride, and capryloyl salicylic acid. This composition provides a gentle yet effective cleansing solution that supports skin health while aligning with consumer preferences for natural and environmentally conscious products.

The micellar aqueous cleansing composition described herein offers unexpected benefits over existing skin cleansing formulations, including those intended for acne treatment and general personal care. Conventional cleansing compositions, including micellar gel compositions, often rely on PEG-based thickeners and sulfate-based surfactants, which can lead to skin irritation and are not favored by consumers seeking more natural formulations. These traditional compositions depend on interactions between surfactants and PEG to maintain thickness, and in the context of micellar gels, to maintain micellar structure; however, without PEG, the micellar structure is typically weak, prone to breakdown, and susceptible to visible liquid phase separation and cloudiness.

In contrast, the inventive micellar aqueous cleansing composition eliminates PEG-based ingredients, specifically PEG-based thickeners, and sulfate-based ingredients, particularly sulfate-based surfactants, and instead incorporates lauryl lactate as a natural thickener alongside cationic conditioning polymers selected from polyquaternium compounds. Unexpectedly, lauryl lactate strengthens the micellar structure, enhancing the solubility and stability of cationic conditioning polymers such as polyquaterniums. As a result, the micellar aqueous cleansing composition demonstrates remarkable visible stability, evidenced by the homogeneous micellar structure and maintained transparency over time and under varied temperature conditions, including stability observed at the two-week mark.

Referencing the examples, the inventors have found that other natural thickeners, such as polysaccharides, do not contribute to stability in the same manner, often resulting in phase separation and haziness due to the destabilization of polyquaterniums caused by their positive charge density. In contrast, lauryl lactate provides a new physical interaction with surfactants, reinforcing the micellar structure and thus improving the solubility and stability of cationic conditioning polymers such as polyquaterniums. While lauryl lactate is known for its use in skin care cosmetics, particularly as a thickener in oil-in-water emulsified compositions, its application in sulfate-free and PEG-free aqueous compositions with cationic conditioning polymers is novel. The surprising discovery that lauryl lactate enhances the solubility and stability of polyquaterniums underscores the innovative nature of the micellar aqueous cleansing composition. In various embodiments, the micellar aqueous cleansing composition is suitable as transparent fluid gels for use in facial cleansers, shampoos, and body washes, providing a gentle yet effective cleansing solution that aligns with consumer preferences for natural and stable formulations. Thus, in various embodiments, the micellar aqueous cleansing composition may be a skin cleanser, shampoo, or body wash.

The micellar aqueous cleansing composition, can comprise, consist of, or consist essentially of the essential elements and limitations of the invention described herein, as well as any additional or optional ingredients, components, or limitations described herein or otherwise useful.

“Cleanser” or “micellar aqueous cleansing composition” as used herein means and refers to any micellar aqueous cleansing composition utilized for application to a keratinous tissue for one or more of cleansing the skin, removal of make-up and the like.

“Cosmetically acceptable” as used herein means and refers to a carrier that is compatible with any keratinous substrate.

As used herein with respect to the foaming property of cleansing formulations, including the micellar aqueous cleansing composition, the term “foaming” means and refers to the capacity of a composition (inventive or comparative) to develop a foam upon rubbing that would be associated with applying and gently scrubbing skin or other keratinous substrates with the formulation. Reference is made to a known testing method for establishing foam formation in vitro employing equipment that is well known in the art, namely analysis using a Krauss DFA100 ™ Dynamic Foam Analyzer, which provides a relative measure of achieved foam formation, as more specifically described herein. Foaming may be described as relative formation of foam density per unit area when compared to other tested formulations under the same conditions. Bubble count using the indicated foam analyzer may be provided. Foaming Capacity, or good foaming or high foaming as used herein relative to foaming on a keratinous substrate, such as facial skin, may be based on a scale from low to high.

“Keratinous substrate” and “keratinous tissue” each includes but is not limited to skin, hair, and nails.

“Skin” as used herein means and refers to skin materials containing keratin such as facial and body skin, scalp, eyebrows, and lips.

“Stable” and “Stability” as used herein means that the micellar aqueous cleansing composition does not show any signs of significant changes in one or more of clarity, color, odor, pH, or viscosity at room temperature and at temperatures in the range from about 25° C. to about 45° C., remaining transparent without haziness and without evidence of precipitation or phase separation. In various embodiments, an inventive composition remains stable at temperatures in the range from about 25° C. to about 45° C., over a time period of at least one month, or at least two months, or longer, or any value, range, or sub-range therebetween. In some embodiments, a micellar aqueous cleansing composition is stable at 25° C. for at least one month, or at least two months, or longer, or any value, range, or sub-range therebetween. In some embodiments, a micellar aqueous cleansing composition is stable at 45° C. for at least one month, or at least two months, or longer, or any value, range, or sub-range therebetween.

Viscosity was measured in the UD units by viscometer Rheomat R180 using an M3 spindle for ~10 minutes at 25° C. Stable inventive compositions demonstrate viscosity in a range from about 15 UD to about 50 UD (M3) based on use of RM180 Rheomat M3 Spindle analysis. In some particular embodiments, the viscosity is in the range from about 20 UD to about 40 UD. Unsatisfactory compositions are deemed unstable and fail at viscosities that fall below 12 UD after exposure to 45° C. for after two months.

The term “clear” or “transparent” with respect to a micellar aqueous cleansing composition indicates that the composition is clear at least to the naked unaided eye, and subject to measurement, has transmittance of at least 80% at a wavelength of 600 nm, for example measured using a Lambda 40 UV-visible spectrometer. The micellar aqueous cleansing composition may have, for example, a transmittance of at least 80%, at least 90%, or at least 95%, or of 100% at a wavelength of 600 nm, measured, for example, using a Lambda 40 UV-visible spectrometer. The term “clear” is interchangeable with the term “transparent” for purposes of the instant disclosure. A human can typically see through a transparent composition, for example, and read the text on the other side of a clear glass or clear plastic bottle containing the composition; however, it may be the case that the “clear” micellar aqueous cleansing composition according to the invention is translucent in that it may not be possible to read text on the other side of a clear glass or clear plastic bottle containing the composition, such that the text may be blurred and difficult or not possible to read, while movement and structure can normally be identified. The term “opaque” with respect to an opaque composition indicates that the composition is not transparent or translucent, i.e., has a transmittance of less than 50% at a wavelength of 600 nm, for example measured using a Lambda 40 UV-visible spectrometer.

### Micellar Aqueous Cleansing Composition

### Natural Thickener

The micellar aqueous cleansing composition of the present invention utilizes at least one natural thickener including at least lauryl lactate. In some embodiments the composition includes only lauryl lactate as the natural thickener and excludes any other thickener. Lauryl lactate is incorporated in the formulation within a concentration range from about 0.1% to about 1% by weight, based on the total weight of the composition. In some embodiments the composition includes other thickeners in addition to lauryl lactate.

In some embodiments, the micellar aqueous cleansing composition includes a natural thickener consisting essentially of lauryl lactate and excludes any other added thickener including any other natural thickener. In some embodiments, the micellar aqueous cleansing composition excludes other natural thickeners such as xanthan gum, sclerotium gum, alginate, hydroxyethylcellulose, Zea Mays starch, carrageenan. Other possible included and excluded thickeners may be found listed herein below as optional included or excluded ingredients.

In various embodiments, lauryl lactate is present in amounts ranging from about 0.1% to about 2%, or from about 0.1% to about 1%, or from about 0.05% to about 0.9%, or from about 0.1% to about 0.8%, or from about 0.4% to about 0.9%, or at about 0.65%, or any suitable combination, sub-combination, range, or sub-range thereof by weight, based on the weight of the micellar aqueous cleansing composition. Thus, lauryl lactate may be present by weight, based on the total weight of the micellar aqueous cleansing composition from about 0.05, 0.1, 0.2, 0.3, 0.4, 0.5, 0.6, 0.7, 0.8, 0.9, 1, 1.5, to about 2 weight percent, including increments and ranges therein and there between.

### Polyquaternium Cationic Polymers

The micellar aqueous cleansing composition of the present invention utilizes at least one polyquaternium cationic polymers.

In some embodiments, the composition includes polyquaternium cationic polymers selected from polyquaternium-22, 39, 47, and 67, each present in the formulation at concentrations of at least 0.5% by weight. The composition may include more than one polyquaternium cationic polymer. In some embodiments, the composition includes at least one polyquaternium cationic polymers selected from polyquaternium-22, 39, 47, and 67, and one or more additional polyquaternium cationic polymers. In some embodiments, the composition includes only one polyquaternium cationic polymer consisting of only one of polyquaternium-22, 39, 47, and 67

In various embodiments, the polyquaternium cationic polymers are present in amounts ranging from about 0.05% to about 1.5%, or from about 0.5% to about 1%, or from about 0.1% to about 0.9%, or about 0.5%, or any suitable combination, sub-combination, range, or sub-range thereof by weight, based on the weight of the micellar aqueous cleansing composition. Thus, polyquaternium cationic polymers may be present by weight, based on the total weight of the micellar aqueous cleansing composition, from about 0.05, 0.1, 0.2, 0.3, 0.4, 0.5, 0.6, 0.7, 0.8, 0.9, 1 to about 1.5 weight percent, including increments and ranges therein and there between.

Generally, the micellar aqueous cleansing composition may include one or more polyquaternium cationic polymers, also referred to as “polyquaterniums.” For example, the hair treatment composition may include polyquaternium-1 (ethanol, 2,2′,2″-nitrilotris-, polymer with 1,4-dichloro-2-butene and N,N,N′,N′-tetramethyl-2-butene-1,4-diamine), polyquaternium-2, (poly[bis(2-chloroethyl) ether-alt-1,3-bis[3-(dimethylamino)propyl]urea]), polyquaternium-4, (hydroxyethyl cellulose dimethyl diallylammonium chloride copolymer; Diallyldimethylammonium chloride-hydroxyethyl cellulose copolymer), polyquaternium-5 (copolymer of acrylamide and quaternized dimethylammoniumethyl methacrylate), polyquaternium-6 (poly(diallyldimethylammonium chloride)), polyquaternium-7 (copolymer of acrylamide and diallyldimethylammonium chloride), polyquaternium-8 (copolymer of methyl and stearyl dimethylaminoethyl ester of methacrylic acid, quaternized with dimethylsulphate), polyquaternium-9 (homopolymer of N,N-(dimethylamino)ethyl ester of methacrylic acid, quaternized with bromomethane), polyquaternium-10 (quaternized hydroxyethyl cellulose), polyquaternium-11 (copolymer of vinylpyrrolidone and quaternized dimethylaminoethyl methacrylate), polyquaternium-12 (ethyl methacrylate/abietyl methacrylate/diethylaminoethyl methacrylate copolymer quaternized with dimethyl sulfate), polyquaternium-13 (ethyl methacrylate/oleyl methacrylate/diethylaminoethyl methacrylate copolymer quaternized with dimethyl sulfate), polyquaternium-14 (trimethylaminoethylmethacrylate homopolymer), polyquaternium-15 (acrylamide-dimethylaminoethyl methacrylate methyl chloride copolymer), polyquaternium-16 (copolymer of vinylpyrrolidone and quaternized vinylimidazole), polyquaternium-17 (adipic acid, dimethylaminopropylamine and dichloroethylether copolymer), polyquaternium-18 (azelanic acid, dimethylaminopropylamine and dichloroethylether copolymer), polyquaternium-19 (copolymer of polyvinyl alcohol and 2,3-epoxypropylamine), polyquaternium-20 (copolymer of polyvinyl octadecyl ether and 2,3-epoxypropylamine), polyquaternium-22 (copolymer of acrylic acid and diallyldimethylammonium chloride), polyquaternium-24 (auaternary ammonium salt of hydroxyethyl cellulose reacted with a lauryl dimethyl ammonium substituted epoxide), polyquaternium-27 (block copolymer of polyquaternium-2 and polyquaternium-17), polyquaternium-28 (copolymer of vinylpyrrolidone and methacrylamidopropyl trimethylammonium), polyquaternium-29 (chitosan modified with propylen oxide and quaternized with epichlorhydrin), polyquaternium-30 (ethanaminium, N-(carboxymethyl)-N,N-dimethyl-2-[(2-methyl-1-oxo-2-propen-1-yl)oxy]-, inner salt, polymer with methyl 2-methyl-2-propenoate), polyquaternium-31 (N,N-dimethylaminopropyl-N-acrylamidine quatemized with diethylsulfate bound to a block of polyacrylonitrile), polyquaternium-32 (poly(acrylamide 2-methacryloxyethyltrimethyl ammonium chloride)), polyquaternium-33 (copolymer of trimethylaminoethylacrylate salt and acrylamide), polyquaternium-34 (copolymer of 1,3-dibromopropane and N,N-diethyl-N′,N′-dimethyl-1,3-propanediamine), polyquaternium-35 (methosulphate of the copolymer of methacryloyloxyethyltrimethylammonium and of methacryloyloxyethyldimethylacetylammonium), polyquaternium-36 (copolymer of N,N-dimethylaminoethylmethacrylate and buthylmethacrylate, quaternized with dimethylsulphate), polyquaternium-37 (poly(2-methacryloxyethyltrimethylammonium chloride)), polyquaternium-39 (terpolymer of acrylic acid, acrylamide and diallyldimethylammonium Chloride), polyquaternium-42 (poly[oxyethylene(dimethylimino)ethylene (dimethylimino)ethylene dichloride]), polyquaternium-43 (copolymer of acrylamide, acrylamidopropyltrimonium chloride, 2-amidopropylacrylamide sulfonate and dimethylaminopropylamine), polyquaternium-44 (3-Methyl-1-vinylimidazolium methyl sulfate-N-vinylpyrrolidone copolymer), polyquaternium-45 (copolymer of (N-methyl-N-ethoxyglycine)methacrylate and N,N-dimethylaminoethylmethacrylate, quaternized with dimethyl sulphate), polyquaternium-46 (terpolymer of vinylcaprolactam, vinylpyrrolidone, and quaternized vinylimidazole), polyquaternium-47 (terpolymer of acrylic acid, methacrylamidopropyl trimethylammonium chloride, and methyl acrylate), and/or polyquaternium-67.

In some instances, the micellar aqueous cleansing composition includes one or more polyquaternium cationic polymers selected from cationic cellulose derivatives, quaternized hydroxyethyl cellulose (e.g., polyquaternium-10), cationic starch derivatives, cationic guar gum derivatives, copolymers of acrylamide and dimethyldiallyammonium chloride (e.g., polyquaternium-7), polyquaterniums, and a mixture thereof. For example, the cationic polymer(s) may be selected from polyquaterniums, for example, polyquaterniums selected from polyquaternium-4, polyquaternium-5, polyquaternium-6, polyquaternium-7, polyquaternium-10, polyquaternium-22, polyquaternium-37, polyquaternium-39, polyquaternium-47, polyquaternium-53, polyquaternium-67 and a mixture thereof. A combination of two or more polyquaterniums can be useful. A particularly preferred and useful cationic polymer is polyquaternium-10.

In certain embodiments, the polyquaterniums may be selected from polyquaternium-1, polyquaternium-2, polyquaternium-3, polyquaternium-4, polyquaternium-5, polyquaternium-6, polyquaternium-7, polyquaternium-8, polyquaternium-9, polyquaternium-10, polyquaternium-11, polyquaternium-12, polyquaternium-13, polyquaternium-14, polyquaternium-15, polyquaternium-16, polyquaternium-17, polyquaternium-18, polyquaternium-19, polyquaternium-20, polyquaternium-21, polyquaternium-22, polyquaternium-23, polyquaternium-24, polyquaternium-25, polyquaternium-26, polyquaternium-27, polyquaternium-28, polyquaternium-29, polyquaternium-30, polyquaternium-40, polyquaternium-41, polyquaternium-42, polyquaternium-43, polyquaternium-44, polyquaternium-45, polyquaternium-46, polyquaternium-47, polyquaternium-48, polyquaternium-49, polyquaternium-50, polyquaternium-51, polyquaternium-52, polyquaternium-53, polyquaternium-54, polyquaternium-55, polyquaternium-56, polyquaternium-57, polyquaternium-58, polyquaternium-59, polyquaternium-60, polyquaternium-61, polyquaternium-62, polyquaternium-63, polyquaternium-64, polyquaternium-65, polyquaternium-66, polyquaternium-67, etc. In some cases, preferred polyquaternium compounds include polyquaternium-10, polyquaternium-11, polyquaternium-67, and a mixture thereof.

The total amount of the at least one polyquaternium cationic polymers in the micellar aqueous cleansing composition, if present, will vary but are typically from about 0.01 to about 3 wt. %, based on the total weight of the micellar aqueous cleansing composition. In further embodiments, the micellar aqueous cleansing composition includes from about 0.01 to about 2 wt. %, about 0.01 to about 1 wt. %, about 0.05 to about 3 wt. %, about 0.05 to about 2 wt. %, about 0.05 to about 1 wt. %, about 0.1 to about 3 wt. %, about 0.1 to about 2 wt. %, about 0.1 to about 1 wt. %, based on the total weight of the micellar aqueous cleansing composition. Preferably, the micellar aqueous cleansing composition includes from about 0.01 to about 3 wt. %, about 0.05 to about 2 wt. %, about 0.1 to about 1 wt. % of the one or more polyquaternium cationic polymers, based on the total weight of the micellar aqueous cleansing composition.

The pH of the micellar aqueous cleansing composition will vary but is typically from about 4 to about 8 or about 4 to about 7. In further embodiments, it is preferable that the pH be acidic or slightly acidic. For example, the pH may be from about 4 to less than 7, about 4.5 to less than 7, about 5 to less than 7, about 5.5 to less than 7, or about 6 to less than 7. In further embodiments, the pH is from about 4 to about 6.5, about 4 to about 6, about 4 to about 5.5, about 4 to about 5, about 4.5 to about 6.5, about 4.5 to about 6, about 4.5 to about 5.5, about 5 to about 6.5, about 5 to about 6, or about 5.5 to about 6.5. Preferably, the micellar aqueous cleansing composition has a pH of about 4 to about 7, more preferably about 4.5 to about 6.5, and even more preferably about 5 to about 6.

Thus, any one of the polyquaternium cationic polymer is present, by weight, based on the total weight of the cosmetic cleansing composition, from about 0.05, 0.06, 0.07, 0.08, 0.09, 0.1, 0.2, 0.3, 0.4, 0.5, 0.6, 0.7, 0.8, 0.9, 1, to about 2 weight percent, including increments and ranges therein and there between.

In the various embodiments, the composition may include an additional catioinic polymer that is not a polyquaternium cationic polymer, each of which additional cationic polymer may be either nature-based or synthetic cationic polymer and may be present in the composition from about 0.05% to about 2%, or from about 0.05% to about 1%, or from about 0.05% to about 1%, or from about 0.1% to about 0.5%, or from about 0.1% to about 0.2% or any suitable combination, sub-combination, range, or sub-range thereof by weight, based on the weight of the cosmetic cleansing composition. One of ordinary skill in the art, however, will appreciate that other ranges are within the scope of the invention.

### Surfactants

### Amphoteric Surfactant

The micellar aqueous cleansing composition may further comprise one or more surfactants. In some embodiments, the micellar aqueous cleansing composition comprises at least one amphoteric surfactant (or zwitterionic surfactant).

Amphoteric surfactants such as cocoamidopropyl hydroxysultaine may be included, present in a range from about 1% to about 20% by weight.

In various embodiments, the at least one amphoteric surfactant, for example, cocoamidopropyl hydroxysultaine, is present in amounts ranging from about 1% to about 20%, or from about 3% to about 10%, or any suitable combination, sub-combination, range, or sub-range thereof by weight, based on the weight of the micellar aqueous cleansing composition. Thus, the at least one amphoteric surfactant, for example, cocoamidopropyl hydroxysultaine, may be present by weight, based on the total weight of the micellar aqueous cleansing composition, from about 1, 2, 3, 4, 5, 6, 7, 8, 9, 10, 15, to about 20 weight percent, including increments and ranges therein and there between.

In some embodiments, the micellar aqueous cleansing composition comprises more than one amphoteric surfactant. In some embodiments, the micellar aqueous cleansing composition may include or excludes any one or more of nonionic, cationic, or anionic surfactants, and in particular embodiments, excludes PEG/POE surfactants. In some embodiments, the micellar aqueous cleansing composition includes one or a combination amphoteric surfactants in addition to cocoamidopropyl hydroxysultaine.

In some embodiments, any additional amphoteric surfactant may be selected from, for example, betaines, alkyl sultaines, alkyl amphoacetates and alkyl amphodiacetates, alkyl amphoproprionates, amphocarboxylates, alkyl betaines, amidoalkyl betaines, amphophosphates, phosphobetaines, pyrophosphobetaines, carboxyalkyl polyamines, amidoalkyl sultaines, salts thereof, or mixtures thereof.

Betaines which can be used in the current compositions include those having the formulas below:

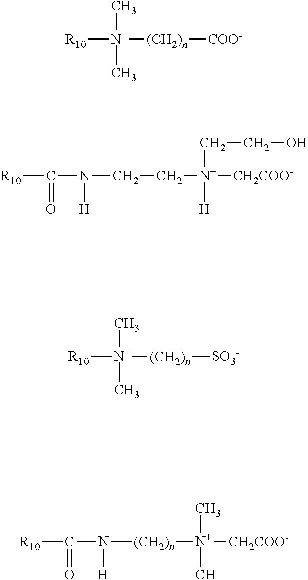

wherein
   R¹⁰ is an alkyl group having 8-18 carbon atoms; and
n is an integer from 1 to 3.

Particularly useful betaines include, for example, coco betaine, cocoamidopropyl betaine, lauryl betaine, laurylhydroxy sulfobetaine, lauryldimethyl betaine, cocoamidopropyl hydroxysultaine, behenyl betaine, capryl/capramidopropyl betaine, lauryl hydroxysultaine, stearyl betaine, and mixtures thereof. Typically, the at least one betaine compound is selected from the group consisting of coco betaine, cocoamidopropyl betaine, behenyl betaine, capryl/capramidopropyl betaine, lauryl betaine, and mixtures thereof, and more typically coco betaine.

Hydroxyl sultaines useful in the compositions of the invention include the following

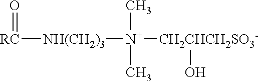

wherein
   R is an alkyl group having 8-18 carbon atoms.

More specific examples include, but are not limited to cocamidopropyl hydroxysultaine, lauryl hydroxysultaine, or mixtures thereof.

Useful alkylamphoacetates include those having the formula

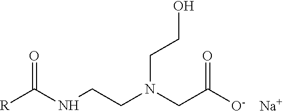

wherein
   R is an alkyl group having 8-18 carbon atoms.
useful alkyl amphodiacetates include those having the formula

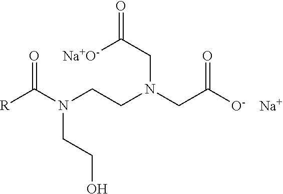

wherein
   R is an alkyl group having 8-18 carbon atoms.

### Alkyl Amphopropionates

Exemplary and non-limiting examples of useful alkyl amphopropionates include cocoamphopropionate, caprylamphopropionate, cornamphopropionate, caproampho-propionate, oleoamphopropionate, isostearoamphopropionate, stearoamphopropionate, lauroamphopropionate, salts thereof, or mixtures thereof.

An amphoteric surfactant may be optionally quaternized secondary or tertiary aliphatic amine derivatives, in which the aliphatic group is a linear or branched chain comprising from 8 to 22 carbon atoms, said amine derivatives containing at least one anionic group, for instance a carboxylate, sulfonate, sulfate, phosphate or phosphonate group.

An amphoteric surfactant may be present in the micellar aqueous cleansing composition in a range from about 0.1% to about 20%, based on the weight of the micellar aqueous cleansing composition.

In various embodiments, an amphoteric surfactant may be present in an amount in the range of from about 0.1% to about 20%, and the total amount of surfactant may be present in a range from about 0.1% to about 20%. In some embodiments, each of the at least one amphoteric surfactant is present in an amount that is from about 1% to about 20%, or from about 1% to about 15%, or from about 3% to about 10%, or from about 5% to about 7%, or any value, range, or sub-range therebetween by weight, based on the weight of the micellar aqueous cleansing composition.

Thus, in various embodiments, an amphoteric surfactant may be present in a composition according to the disclosure from 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19 to about 20 percent, including increments and ranges therein and there between.

### Anionic Surfactant

In some embodiment, the composition includes at least one anionic surfactant.

In some embodiments, the composition may include anionic surfactants, for example, sodium methyl cocoyl taurate, present from about 0.1% to about 1.5% by weight.

In various embodiments, the at least one anionic surfactant, for example, sodium methyl cocoyl taurate, is present in amounts ranging from about 0.1% to about 1.5%, or from about 0.5% to about 1.2%, or about 0.9%, or any suitable combination, sub-combination, range, or sub-range thereof by weight, based on the weight of the micellar aqueous cleansing composition. Thus, In various embodiments, the at least one anionic surfactant, for example, sodium methyl cocoyl taurate, may be present by weight, based on the total weight of the micellar aqueous cleansing composition, from about 0.1, 0.2, 0.3, 0.4, 0.5, 0.6, 0.7, 0.8, 0.9, 1, 1.2, to about 1.5 weight percent, including increments and ranges therein and there between.

In some embodiments, the micellar aqueous cleansing composition comprises more than one anionic surfactant. In some embodiments, the micellar aqueous cleansing composition may include or excludes any one or more of amphoteric, nonionic, or cationic surfactants, and in particular embodiments, excludes PEG/POE surfactants. In some embodiments, the micellar aqueous cleansing composition includes one or a combination anionic surfactants in addition to sodium methyl cocoyl taurate.

In some embodiments, any additional anionic surfactant may be selected from non-sulfate anionic surfactants.

When present, the total amount of non-sulfate anionic surfactants in the micellar aqueous cleansing compositions can range from about 4% to about 30%, based on the total weight of the micellar aqueous cleansing composition. In some cases, the total amount of non-sulfate anionic surfactants in the micellar aqueous cleansing composition may be from about 4% to about 25%, or from about 5% to about 25%, or from about 5% to about 23%, or from about 6% to about 24%, or from about 6% to about 25%, or from about 7% to about 25%, or from about 8% to about 25%, or from about 10% to about 23%, or any suitable combination, sub-combination, range, or sub-range thereof by weight, based on the weight of the micellar aqueous cleansing composition based.

In various embodiments, the total amount of non-sulfate anionic surfactants in the micellar aqueous cleansing compositions, if present, is typically at about 6, 6.5, 7, 7.5, 8, 8.5, 8.8, 9, 9.5, 10, 10.5, 11, 11.4, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, to about 25, weight percent based on the total weight of the micellar aqueous cleansing composition, including increments and ranges therein and there between.

In some embodiments, any additional anionic surfactant may be selected from alkyl sulfonates, alkyl sulfosuccinates, alkyl sulfoacetates, acyl isethionates, alkoxylated monoacids, acyl amino acids such as acyl taurates, acyl glycinates, acyl glutamates, acyl sarcosinates, salts thereof, or mixtures thereof. Non-limiting examples of useful non-sulfate anionic surfactants are provided below.

### Acyl Isethionates

Non-limiting examples of useful acyl isethionates and their salts include those of formula (III) and (IV):

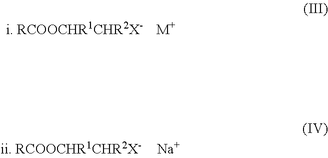

wherein:
   R, R¹, and R² are each independently chosen from H or an alkyl chain having 1-24 carbon atoms, said chain being saturated or unsaturated, linear or branched;
   X is COO⁻ or SO₃⁻; and
   M is any suitable cation.

Although the cation may be chosen from any suitable cation as in formula (III), including, for example, alkali metal ion such as sodium or potassium, ammonium ions, or alkanolammonium ions such as monoethanolammonium or triethanolammonium ions, sodium is an cation, as in formula (IV). In various embodiments, RCO— represents the coconut acid moiety. Non-limiting examples of acyl isethionates include sodium cocoyl isethionate, sodium lauroyl isethionate, sodium lauroyl methyl isethionate, and sodium cocoyl methyl isethionate.

### Acyl Sarcosinates

Non-limiting examples of acyl sarcosinates and their salts include potassium lauroyl sarcosinate, potassium cocoyl sarcosinate, sodium cocoyl sarcosinate, sodium lauroyl sarcosinate, sodium myristoyl sarcosinate, sodium oleoyl sarcosinate, sodium palmitoyl sarcosinate, and ammonium lauroyl sarcosinate.

In some embodiments, the total amount of acyl sarcosinates in the micellar aqueous cleansing composition, if present, is from about 0.5 to about 10%, all amounts by weight based on the weight of the micellar aqueous cleansing composition. In some instance, the total amount of acyl sarcosinates in the micellar aqueous cleansing composition is from about 0.5 to about 10%, about 1 to about 8%, about 1.5 to about 7%, about 1.75 to about 6%, 2 to about 5%, about 2 to about 4%, about 2.5 to about 4%, about 2.5 to about 3.5%, or about 2.7 to about 3%, all amounts by weight based on the weight of the micellar aqueous cleansing composition, including all ranges and subranges therebetween.

### Alkyl Sulfonates

Useful alkyl sulfonates and their salts include alkyl aryl sulfonates, primary alkane disulfonates, alkene sulfonates, hydroxyalkane sulfonates, alkyl glyceryl ether sulfonates, alpha-olefinsulfonates, sulfonates of alkylphenolpolyglycol ethers, alkylbenzenesulfonates, phenvlalkanesulfonates, alpha-olefinsulfonates, olefin sulfonates, alkene sulfonates, hydroxyalkanesulfonates and disulfonates, secondary alkanesulfonates, paraffin sulfonates, ester sulfonates, sulfonated fatty acid glycerol esters, and alpha-sulfo fatty acid methyl esters including methyl ester sulfonate.

In some instances, an alkyl sulfonate of formula (V) is particularly useful:

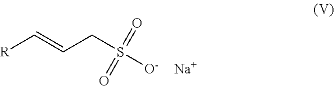

wherein R is selected from H or alkyl chain that has 1-24 carbon atoms, 6-24 carbon atoms, more, 8 to 20 carbon atoms, said chain being saturated or unsaturated, linear or branched, substituted or unsubstituted. Sodium is shown as the cation in the above formula (V) but the cation may be an alkali metal ion such as sodium or potassium, ammonium ions, or alkanolammonium ions such as monoethanolammonium or triethanolammonium ions.

In some instances, the alkyl sulfonate(s) are selected from C₈-C₁₆ alkyl benzene sulfonates, C₁₀-C₂₀ paraffin sulfonates, C₁₀-C₂₄ olefin sulfonates, salts thereof, or mixtures thereof. A non-limiting but particularly useful example of a C₁₀-C₂₄ olefin sulfonate that can be used in the instant compositions is sodium C₁₄₋₁₆ olefin sulfonate.

### Alkyl Sulfosuccinates

Non-limiting examples of useful alkyl sulfosuccinates and their salts include those of formula (VI):

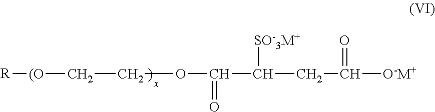

wherein:
1. R is a straight or branched chain alkyl or alkenyl group having 10 to 22 carbon atoms, 10 to 20 carbon atoms;
2. x is a number that represents the average degree of ethoxylation, and can range from 0 to about 5, from 0 to about 4, and most from about 2 to about 3.5; and
3. M, which can be the same or different, is chosen from any suitable monovalent cation.

Preferred cations are alkali metal ions such as sodium or potassium, ammonium ions, or alkanolammonium ions such as monoethanolammonium or triethanol-ammonium ions.

Non-limiting examples of alkyl sulfosuccinates salts include disodium oleamido MIPA sulfosuccinate, disodium oleamido MEA sulfosuccinate, disodium lauryl sulfosuccinate, disodium laureth sulfosuccinate, diammonium lauryl sulfosuccinate, diammonium laureth sulfosuccinate, dioctyl sodium sulfosuccinate, disodium oleamide MEA sulfosuccinate, sodium dialkyl sulfosuccinate, or mixtures thereof.

### Alkyl Sulfoacetates

Non-limiting examples of alkyl sulfoacetates and their salts include, for example, alkyl sulfoacetates such as C4-C18 fatty alcohol sulfoacetates and/or salts thereof, for example, sodium lauryl sulfoacetate. Useful cations for the salts include alkali metal ions such as sodium or potassium, ammonium ions, or alkanolammonium ions such as monoethanolammonium or triethanolammonium ions.

### Alkoxylated Monoacids

Non-limiting examples of alkoxylated monoacids include compounds corresponding to formula (VII):

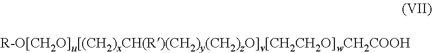

wherein:
1. R is a hydrocarbon radical containing from about 6 to about 40 carbon atoms;
2. R′ represents hydrogen or alkyl;
3. u, v, and w, which may be identical or different, independently represent numbers from 0 to 60;
4. x, y, and z, which may be identical or different, independently represent numbers from 0 to 13; and
5. the sum of x+y+z>0.

Compounds corresponding to formula (VII) can be obtained by alkoxylation of alcohols R—OH with ethylene oxide as the sole alkoxide or with several alkoxides and subsequent oxidation. The numbers u, v, and w each represent the degree of alkoxylation. Whereas, on a molecular level, the numbers u, v, and w and the total degree of alkoxylation can only be integers, including zero, on a macroscopic level they are mean values in the form of broken numbers.

In formula (VII), R is linear or branched, acyclic or cyclic, saturated or unsaturated, aliphatic or aromatic, substituted or unsubstituted. Typically, R is a linear or branched, acyclic C6-C40 alkyl or alkenyl group or a C1-C40 alkyl phenyl group, more typically a C8-C22 alkyl or alkenyl group, or a C4-C18 alkyl phenyl group, and even more typically a C12-C18 alkyl group or alkenyl group or a C6-C16 alkyl phenyl group. Further, u, v, w, independently of one another, are typically chosen from a number ranging from 2 to 20, more typically a number ranging from 3 to 17, and most typically a number ranging from 5 to 15. Further still, x, y, z, independently of one another, are typically chosen from a number ranging from 0 to 13, more typically a number ranging from 1 to 10, and most typically a number ranging from 2 to 8.

Suitable alkoxylated monoacids include, but are not limited to: Butoxynol-5 Carboxylic Acid, Butoxynol-19 Carboxylic Acid, Capryleth-4 Carboxylic Acid, Capryleth-6 Carboxylic Acid, Capryleth-9 Carboxylic Acid, Ceteareth-25 Carboxylic Acid, Coceth-7 Carboxylic Acid, C9-11 Pareth-6 Carboxylic Acid, C11-15 Pareth-7 Carboxylic Acid, C12-13 Pareth-5 Carboxylic Acid, C12-13 Pareth-8 Carboxylic Acid, C12-13 Pareth-12 Carboxylic Acid, C12-15 Pareth-7 Carboxylic Acid, C12-15 Pareth-8 Carboxylic Acid, C14-15 Pareth-8 Carboxylic Acid, Deceth-7 Carboxylic Acid, Laureth-3 Carboxylic Acid, Laureth-4 Carboxylic Acid, Laureth-5 Carboxylic Acid, Laureth-6 Carboxylic Acid, Laureth-8 Carboxylic Acid, Laureth-10 Carboxylic Acid, Laureth-11 Carboxylic Acid, Laureth-12 Carboxylic Acid, Laureth-13 Carboxylic Acid, Laureth-14 Carboxylic Acid, Laureth-17 Carboxylic Acid, PPG-6-Laureth-6 Carboxylic Acid, PPG-8-Steareth-7 Carboxylic Acid, Myreth-3 Carboxylic Acid, Myreth-5 Carboxylic Acid, Nonoxynol-5 Carboxylic Acid, Nonoxynol-8 Carboxylic Acid, Nonoxynol-10 Carboxylic Acid, Octeth-3 Carboxylic Acid, Octoxynol-20 Carboxylic Acid, Oleth-3 Carboxylic Acid, Oleth-6 Carboxylic Acid, Oleth-10 Carboxylic Acid, PPG-3-Deceth-2 Carboxylic Acid, Capryleth-2 Carboxylic Acid, Ceteth-13 Carboxylic Acid, Deceth-2 Carboxylic Acid, Hexeth-4 Carboxylic Acid, Isosteareth-6 Carboxylic Acid, Isosteareth-11 Carboxylic Acid, Trudeceth-3 Carboxylic Acid, Trideceth-6 Carboxylic Acid, Trideceth-8 Carboxylic Acid, Trideceth-12 Carboxylic Acid, Trideceth-3 Carboxylic Acid, Trideceth-4 Carboxylic Acid, Trideceth-7 Carboxylic Acid, Trideceth-15 Carboxylic Acid, Trideceth-19 Carboxylic Acid, Undeceth-5 Carboxylic Acid, or mixtures thereof.

### Acyl Amino Acids

In some embodiments, any additional anionic surfactant may be selected from acyl amino acids that include, but are not limited to, amino acid surfactants based on alanine, arginine, aspartic acid, glutamic acid, glycine, isoleucine, leucine, lysine, phenylalanine, serine, tyrosine, valine, sarcosine, threonine, and taurine. The most common cation associated with the acyl amino acid can be sodium or potassium. Alternatively, the cation can be an organic salt such as triethanolamine (TEA) or a metal salt.

Non-limiting examples of useful acyl amino acids include those of formula (VIII):

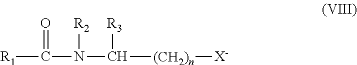

wherein:
   R¹, R², and R³ are each independently selected from H or an alkyl chain having 1-24 carbon atoms, said chain being saturated or unsaturated, linear or branched, substituted or unsubstituted;
   n ranges from 0 to 30; and
   X is COO⁻ or SO₃⁻.

In various embodiments, RCO— represents the coconut acid moiety. A non-limiting example is sodium cocoyl glycinate.

### Acyl Taurates

Non-limiting examples of acyl taurates include those of formula (IX):

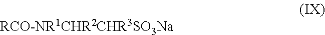

wherein R, R¹, R², and R³ are each independently selected from H or an alkyl chain having from 1-24 carbon atoms, such as from 6-20 carbon atoms, or from 8-16 carbon atoms, said chain being saturated or unsaturated, linear or branched, substituted or unsubstituted.

In various embodiments, RCO— represents the coconut acid moiety. Non-limiting examples of acyl taurate salts include sodium cocoyl taurate and sodium methyl cocoyl taurate.

### Acyl Glycinates

Non-limiting examples of useful acyl glycinates include those of formula (X):

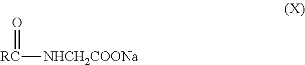

wherein R is an alkyl chain of 8 to 16 carbon atoms. Sodium is shown as the cation in the above formula (X), but the cation may be any alkali metal ion such as sodium or potassium, ammonium ions, or alkanolammonium ions such as monoethanolammonium or triethanolammonium ions.

Non-limiting examples of acyl glycinates include sodium cocoyl glycinate, sodium lauroyl glycinate, sodium myristoyl glycinate, potassium lauroyl glycinate, and potassium cocoyl glycinate, and in particular sodium cocoyl glycinate.

### Acyl Glutamates

Non-limiting examples of useful acyl glutamates include those of formula (XI):

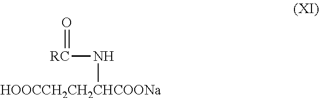

wherein R is an alkyl chain of 8 to 16 carbon atoms. Sodium is shown as the cation in the above formula (XI) but the cation may be any alkali metal ion such as sodium or potassium, ammonium ions, or alkanolammonium ions such as monoethanolammonium or triethanolammonium ions.

Non-limiting examples of acyl glutamates include dipotassium capryloyl glutamate, dipotassium undecylenoyl glutamate, disodium capryloyl glutamate, disodium cocoyl glutamate, disodium lauroyl glutamate, disodium stearoyl glutamate, disodium undecylenoyl glutamate, potassium capryloyl glutamate, potassium cocoyl glutamate, potassium lauroyl glutamate, potassium myristoyl glutamate, potassium stearoyl glutamate, potassium undecylenoyl glutamate, sodium capryloyl glutamate, sodium cocoyl glutamate, sodium lauroyl glutamate, sodium myristoyl glutamate, sodium olivoyl glutamate, sodium palmitoyl glutamate, sodium stearoyl glutamate, sodium undecylenoyl glutamate, triethanolamine mono-cocoyl glutamate, triethanolamine lauroylglutamate, and disodium cocoyl glutamate.

In some embodiments, any additional anionic surfactant may be selected from isethionates, their salts, or mixtures thereof.

In an embodiment, the one or more acyl isethionates, their salts, or mixtures thereof are selected from sodium cocoyl isethionate, sodium lauroyl methyl isethionate, sodium cocoyl methyl isethionate, sodium lauroyl isethionate, or mixtures thereof.

In an embodiment, the non-sulfate anionic surfactants of the micellar aqueous cleansing compositions of the present disclosure are selected from one or more acyl sarcosinates, their salts, or mixtures thereof.

In an embodiment, the one or more acyl sarcosinates, their salts, or mixtures thereof are selected from sodium cocoyl sarcosinate, sodium lauroyl sarcosinate, potassium lauroyl sarcosinate, potassium cocoyl sarcosinate, sodium myristoyl sarcosinate, sodium oleoyl sarcosinate, sodium palmitoyl sarcosinate, ammonium lauroyl sarcosinate, or mixtures thereof.

For example, two or more, such as three or more non-sulfate anionic surfactants may be selected from acyl isethionates, acyl sarcosinates, alkyl sulfonates, alkyl sulfosuccinates, alkyl sulfoacetates, alkoxylated monoacids, and acyl amino acids such as acyl taurates, acyl glycinates, acyl glutamates, salts thereof, or mixtures thereof.

### Sulfate Anionic Surfactants

Optionally, anionic surfactants may be chosen from sulfate anionic surfactants. Such sulfate anionic surfactants may be chosen from, for example, alkyl sulfates, alkyl ether sulfates, and/or salts thereof.

By way of example, alkyl sulfates may include C₈₋₁₈ alkyl sulfates, more C₁₂₋₁₈ alkyl sulfates, such as in the form of a salt with a solubilizing cation such as sodium, potassium, ammonium, or substituted ammonium. Examples include but are not limited to sodium lauryl sulfate (SLS) and sodium dodecyl sulfate (SDS).

As a further example, alkyl ether sulfates may include those having the formula:

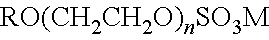

wherein:
   R is an alkyl or alkenyl having from 8 to 18 (12 to 18) carbon atoms;
   n is a number having an average value of greater than at least 0.5, between 1 and 3, more between 2 and 3; and
   M is a solubilizing cation such as sodium, potassium, ammonium or substituted ammonium.

An example of a useful alkyl ether sulfate is sodium lauryl ether sulfate (SLES).

In some instances, useful alkyl sulfate salts and alkyl ether sulfate salts include those having the formulas (XII and XIII):

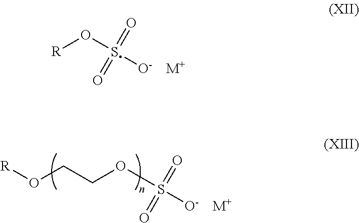

wherein:
   R is an alkyl chain having from 6 to 24 carbon atoms, 8 to 18 carbon atoms, and more 12 to 18 carbon atoms;
   M is a solubilizing cation such as alkali metal ions such as sodium or potassium, ammonium ions, or alkanolammonium ions such as monoethanolammonium or triethanolammonium ions; and
   n is an integer from 0 to 3.

In some embodiments, micellar aqueous cleansing composition according to the disclosure does not require silicones and/or sulfate-based anionic surfactants. Thus, any one or more (or all) of these may optionally be excluded from the micellar aqueous cleansing compositions. In other words, the micellar aqueous cleansing compositions may be free or essentially free of silicones and/or sulfate-based anionic surfactants. Nonetheless, in some instances, one or more silicones and/or one or more sulfate-based surfactants may optionally be included in at least certain embodiments of the micellar aqueous cleansing compositions.

Thus, in various embodiments, one or more additional anionic surfactant may be present in a composition according to the disclosure from 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 120, 21, 22, 23, 24, 25, 26, 27,28,29, 30, 3, 32, 33, 34, 35, 36, 37, 38, 39, 40, 41, 42, 43, 44, 45, 46, 47, 48, 49 to about 50 percent, including increments and ranges therein and there between.

In some embodiments, any additional anionic surfactant may be selected from anionic surfactant having a carboxyl group selected from amino acid surfactants or biosurfactants, and combinations thereof.

In some embodiments, the at least one anionic surfactant having a carboxyl group is selected from the group consisting of amino acid surfactants, biosurfactants, and combinations thereof.

In some embodiments, the at least one anionic surfactant having a carboxyl group is selected from the group consisting of sarcosinates, glycinates, alaninates, glutamates, glycolipids, and combinations thereof.

In some embodiments, the at least one biosurfactant is a glycolipid selected from the group consisting of rhamnolipids, trehalolipids, sophorolipids, mannosylerythritol lipids, and combinations thereof.

In some embodiments, the at least one anionic surfactant having a carboxyl group comprises an amino acid surfactant selected from the group consisting of sodium cocoyl alaninate, sodium cocoyl glycinate, sodium lauroyl sarcosinate, sodium cocoyl glutamate, and combinations thereof.

In some embodiments, the anionic surfactant having a carboxyl group includes at least one glycolipid selected from rhamnolipids. Rhamnolipids are glycolipids produced by various bacterial species. They consist of one rhamnose fragment (mono-rhamnolipid) or of two rhamnose fragments (di-rhamnolipid) linked by a glycosidic bond to one, two or three chains of β-hydroxylated fatty acids linked to one another by an ester bond. Use may be made, as rhamnolipid, of the one sold under the name Rheance One by Evonik (INCI name: glycolipids).

### Water

In the various embodiments, the micellar aqueous cleansing composition includes water.

In various embodiments, the micellar aqueous cleansing composition comprises from about 40% to about 90% water, and In some embodiments, from about 50% to about 85% water, and In some embodiments, from about 60% to about 75% water, including increments and all ranges and subranges therein and there between, by weight, based on the total weight of the micellar aqueous cleansing composition. One of ordinary skill in the art, however, will appreciate that other ranges are within the scope of the invention. Thus, water is present, by weight, based on the total weight of the micellar aqueous cleansing composition, from about 40, 45, 50, 55, 60, 65, 66, 67, 68, 69, 70, 71, 72, 73, 74, 75, 80, 85, to about 90 weight percent, including increments and all ranges and subranges therein and there between.

The water used in the micellar aqueous cleansing composition may be sterile demineralized water and/or a floral water such as rose water, cornflower water, chamomile water or lime water, and/or a natural thermal or mineral water such as, for example: water from Vittel, water from the Vichy basin, water from Uriage, water from La Roche Posay, water from La Bourboule, water from Enghien-les-Bains, water from Saint Gervais-les-Bains, water from Neris-les-Bains, water from Allevar-les-Bains, water from Digne, water from Maizieres, water from Neyrac-les-Bains, water from Lons-le-Saunier, water from Eaux Bonnes, water from Rochefort, water from Saint Christau, water from Les Fumades, water from Tercis-les-Bains or water from Avene. The water phase may also comprise reconstituted thermal water, that is to say a water comprising trace elements such as zinc, copper, magnesium, etc., reconstituting the characteristics of a thermal water.

### Exclusions

The micellar aqueous cleansing composition excludes PEG-150 pentaerythrityl tetrastearate, PEG-6 caprylic/capric glycerides, and other similar PEG-based ingredients. These exclusions are intended to align the composition with consumer preferences for formulations that are free from synthetic polymers and potentially harsh surfactants, thereby enhancing the product's appeal to environmentally and health-conscious consumers. Other possible excluded ingredients, including PEG-based and sulfate-based ingredients are described further, herein below.

In summary, the micellar aqueous cleansing composition provides a balanced formulation that leverages natural thickeners and state-of-the-art cationic polymers to deliver a product that is both effective and gentle on the skin, with a focus on maintaining transparency and stability without the use of PEG and sulfate ingredients.

### Additional Ingredients

The micellar aqueous cleansing composition may also incorporate various additional ingredients that enhance its properties and performance.

In some embodiments, the micellar aqueous cleansing composition may include one or more of other additional ingredients (also referred to as an additive or active) selected from the group consisting of skin care actives, humectants, conditioning agents, thickeners, viscosity adjusters, cooling agents, fillers, antimicrobials, preservatives, pH adjusters, chelating agents, and combinations thereof.

In some particular embodiments, ingredients such as hexylene glycol, sodium hydroxide, menthol, zinc gluconate, tetrasodium glutamate diacetate, sodium benzoate, glycerin, salicylic acid, sodium chloride, and capryloyl salicylic acid can be included.

In some particular embodiments, the micellar aqueous cleansing composition comprises additional ingredients that include hexylene glycol (0.5%), sodium hydroxide (0.4%), menthol (0.1%), zinc gluconate (0.2%), tetrasodium glutamate diacetate (0.0475%), sodium benzoate (0.001%), glycerin (5.0%), sodium chloride (2.12%), and capryloyl salicylic acid (0.05%).

In some particular embodiments, the micellar aqueous cleansing composition comprises additional ingredients that include hexylene glycol (0.50%), sodium hydroxide (0.40%), menthol (0.10%), zinc gluconate (0.20%), tetrasodium glutamate diacetate (0.0475%), sodium benzoate (0.001%), glycerin (5.00%), salicylic acid (2.00%), sodium chloride (2.12%), and capryloyl salicylic acid (0.05%).

In various embodiments, additional ingredients may be present in amounts ranging from about 0.05% to about 5%, or from about 0.1% to about 3%, or from about 0.5% to about 2%, or any suitable combination, sub-combination, range, or sub-range thereof by weight, based on the weight of the micellar aqueous cleansing composition. Thus, additional ingredients may be present by weight, based on the total weight of the micellar aqueous cleansing composition, from about 0.05, 0.1, 0.2, 0.3, 0.4, 0.5, 1, 2, 3, 4, to about 5 weight percent, including increments and ranges therein and there between.

In some embodiments, the micellar aqueous cleansing composition includes at least one additive used in the cosmetics field which does not adversely affect the properties of the micellar aqueous cleansing composition according to the invention, such as, water soluble solvents, oils, preservatives, as well as fragrances, pH adjusters (citric acid, sodium chloride, lactic acid); neutralizing or pH-adjusting agents (e.g., triethylamine (TEA) and sodium hydroxide, Trisodium ethylenediamine disuccinate, and combinations thereof), other cosmetically acceptable additives, such as but not limited to, pearlescent agents, silica, and coloring materials; essential oils; fruit extracts, for example, Pyrus Malus (Apple) Fruit Extract, and Aloe Barbadensis Leaf Juice Powder; actives (for example, hydroxyacetophenone); vitamins (for example, vitamin A, beta carotene, tocopherol/vitamin E, panthenol, retinol, resveratrol, vitamin C, niacinamide, derivatives thereof, and combinations thereof); hyaluronic acid in the form of hydrolyzed hyaluronic acid or sodium hyaluronate; coloring materials/pigments; essential oils; antioxidants(phenolic compounds, such as chalcones, flavones, flavanones, flavanols, flavonols, dihydroflavonols, isoflavonoids, neoflavonoids, catechins, anthocyanidins, tannins, lignans, aurones, stilbenoids, curcuminoids, alkylphenols, betacyanins, capsacinoids, hydroxybenzoketones, methoxyphenols, naphthoquinones, and phenolic terpenes, resveratrol, curcumin, pinoresinol, ferulic acid, hydroxytyrosol, cinnamic acid, caffeic acid, p-coumaric acid, baicalin (Scutellaria Baicalensis root extract), pine bark extract (Pinus Pinaster bark/bud extract), ellagic acid); vitamins and vitamin derivatives, such as calcium pantothenate, tocopherol and ascorbic acid; hydroxy acids; citric acid, sodium citrate, sodium chloride; neutralizing, chelating or pH-adjusting agents (for example, triethylamine (TEA), trisodium ethylenediamine disuccinate, EDTA, and sodium hydroxide); powders, fragrances, dyes, pigments; organic or mineral UV filters; conditioning agents such as C12-15 Alkyl lactate and C12-15 Alcohols; lauryl lactate; Thickeners, for example Ammonium polyacryloyldimethyl taurate, Propylene glycol and PEG-55 propylene glycol oleate; viscosity adjusters, for example, Sodium chloride and Hexylene Glycol; cooling agents such as menthol; fillers, for example Kaolin; or any combination thereof. Although the optional additives are given as examples, it will be appreciated that other optional components compatible with cosmetic applications known in the art may be used. Additives may also include humectants selected from glycerin, glycerol, butylene glycol, propylene glycol/propanediol, isoprene glycol, dipropylene glycol, hexylene glycol and polyethylene glycols, monoethylene glycol, diethylene glycol, triethylene glycol, diethylene glycol, hexylene glycol, glycol ethers such as monopropylene, dipropylene and tripropylene glycol alkyl(C1-C4)ethers, squalane, triacetin, sugars, such as glucose, xylitol, maltitol, sorbitol, sucrose pentaerythritol, inositol, pyrrolidone carboxylic acid, lactic acid, lithium chloride, acetamide MEA, sodium lactate, urea, dicyanamide, hyaluronic acid, aloe vera, honey, seaweed extract, or combinations thereof.

In some embodiments, there may be one or more actives selected from, for example, preservatives/anti-microbials (for example, chlorphenesin, salicylic acid, phenoxyethanol, potassium sorbate, and caprylyl glycol); actives (for example, hydroxyacetophenone); vitamins (for example, vitamin A, beta carotene, tocopherol/vitamin E, panthenol, retinol, resveratrol, vitamin C, niacinamide, derivatives thereof, and combinations thereof); hyaluronic acid in the form of hydrolyzed hyaluronic acid or sodium hyaluronate; coloring materials/pigments; essential oils; antioxidants(phenolic compounds, such as chalcones, flavones, flavanones, flavanols, flavonols, dihydroflavonols, isoflavonoids, neoflavonoids, catechins, anthocyanidins, tannins, lignans, aurones, stilbenoids, curcuminoids, alkylphenols, betacyanins, capsacinoids, hydroxybenzoketones, methoxyphenols, naphthoquinones, and phenolic terpenes, resveratrol, curcumin, pinoresinol, ferulic acid, hydroxytyrosol, cinnamic acid, caffeic acid, p-coumaric acid, baicalin (Scutellaria Baicalensis root extract), pine bark extract (Pinus Pinaster bark/bud extract), ellagic acid); vitamins and vitamin derivatives, such as calcium pantothenate, tocopherol and ascorbic acid; hydroxy acids; citric acid, sodium citrate, sodium chloride; neutralizing, chelating or pH-adjusting agents (for example, triethylamine (TEA), trisodium ethylenediamine disuccinate, EDTA, and sodium hydroxide); powders, fragrances, dyes, pigments; organic or mineral UV filters; and combinations thereof.

In the various embodiments, the amount of one or more additives, alone or in combination, present in the micellar aqueous cleansing composition can be present in a range from about 0.0001% to about 20%, and In some embodiments, from about 0.005% to about 0.01%, and In some embodiments, from about 0.01% to about 0.1%, and In some embodiments, from about 0.15% to about 5%, and In some embodiments, from about 0.40% to about 4%, and In some embodiments, from about 0.5% to about 2.5%, and In some embodiments, from about 0.1% to about 0.5% and In some embodiments, from about 1% to about 2%, or any suitable combination, sub-combination, range, or sub-range thereof by weight, based on the total weight of the the micellar aqueous cleansing composition. One of ordinary skill in the art, however, will appreciate that other ranges are within the scope of the invention.

Thus, any one or a combination of additional ingredients may be present, by weight, based on the total weight of the micellar aqueous cleansing composition, each one or the combination present from about 0.0001, 0.0005, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1.0, 2, 3, 4, 5, 6, 7, 8, 9, 10, 11, 12, 13, 14, 15, 16, 17, 18, 19 to about 20 weight percent, including increments and ranges therein and there between.

### Water-Soluble Solvents

In some embodiments, the composition may include as additional ingredients at least one water-soluble solvent. The term “water-soluble solvent” is interchangeable with the term “water-miscible solvent” and means a compound that is liquid at 25° C. and at atmospheric pressure (760 mmHg), and it has a solubility of at least 50% in water under these conditions. In some cases, the water-soluble solvent has a solubility of at least 60%, 70%, 80%, or 90% in water under these conditions. Non-limiting examples of water-soluble solvents include, for example, glycerin, alcohols (for example, C1-C30, C1-C15, C1-C10, or C1-C4 alcohols), polyols, glycols, and combinations thereof.

As examples of organic solvents, non-limiting mentions can be made of monoalcohols and polyols such as ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, propane diol, and glycerin. The organic solvents can be volatile or non-volatile compounds.

Further non-limiting examples of water-soluble solvents include alkanols (polyhydric alcohols such as glycols and polyols) such as glycerin, 1,2,6-hexanetriol, trimethylolpropane, ethylene glycol, propylene glycol, diethylene glycol, butylene glycol, hexylene glycol, triethylene glycol, tetraethylene glycol, pentaethylene glycol, dipropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, 2-butene-1,4-diol, 2-ethyl-1,3-hexanediol, 2-methyl-2,4-pentanediol, (caprylyl glycol), 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, and combinations thereof.

In some cases, a water-soluble solvent may be selected from the group consisting of pentylene glycol, glycerin, and combinations thereof.

In the various embodiments total amount of the at least one water-soluble solvent, when present, may vary, is from about 0.5% to about 25%, or from about 0.5% to about 20%, or from about 1% to about 20%, or from about 1% to about 10%, or from about 2% to about 5% or any suitable combination, sub-combination, range, or sub-range thereof by weight, based on the weight of the micellar aqueous cleansing composition. One of ordinary skill in the art, however, will appreciate that other ranges are within the scope of the invention.

Thus, any one water-soluble solvent, when present, is present, by weight, based on the total weight of the micellar aqueous cleansing composition, from about 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24 to about 25 weight percent, including increments and ranges therein and there between.

The pH of the micellar aqueous cleansing composition is not limited but is generally from 2 and 12, and In some embodiments, is one of from 3 and 11, and from 4 and 9, and from 4.5 and 6, and In some embodiments, is about pH 5.

The pH can be adjusted to the desired value by addition of a base (organic or inorganic) to the micellar aqueous cleansing composition, for example ammonia or a primary, secondary or tertiary (poly)amine, such as monoethanolamine, diethanolamine, triethanolamine, isopropanolamine or 1,3-propanediamine, or alternatively by addition of an inorganic or organic acid, advantageously a carboxylic acid, such as, for example, citric acid.

In some embodiments, the micellar aqueous cleansing composition excludes alcohols, for example, monoalcohols such as monohydric C₁-C₈ alcohols such as ethanol, propanol, butanol, isopropanol, isobutanol, and benzyl alcohol, and phenylethyl alcohol.

### Oils

In some embodiments, the composition may include as additional ingredients one or more oils. In some embodiments, the micellar aqueous cleansing composition is essentially free, free of or devoid of oils.

In some examples, the oils may be chosen from hydrocarbon-based oils. For example, the hydrocarbon-based oil may be a saturated hydrocarbon, an unsaturated hydrocarbon, lipids, triglycerides, a natural oil, and/or a synthetic oil. In some embodiments, the micellar aqueous cleansing composition may include a synthetic oil selected from the group consisting of hydrogenated polyisobutene and hydrogenated polydecene. A hydrocarbon-based oil may be a non-volatile hydrocarbon-based, such as:
(i) hydrocarbon-based oils of plant origin, such as glyceride triesters, which are generally triesters of fatty acids and of glycerol, the fatty acids of which can have varied chain lengths from C4 to C24, it being possible for these chains to be saturated or unsaturated and linear or branched; these oils are in particular wheat germ oil, sunflower oil, grape seed oil, sesame oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin seed oil, marrow oil, blackcurrant oil, evening primrose oil, millet oil, barley oil, quinoa oil, rye oil, safflower oil, candlenut oil, passionflower oil, musk rose oil, and essential oils, such as Helianthus Annuus Seed Oil, Lavandula Angustifolia (lavender) Oil, Mentha Piperita Oil, Rosmarinus Officinalis (rosemary) Leaf Oil Pelargonium Graveolens flower oil, Citrus Aurantium Dulcis (orange) peel oil, Menthe Viridis (spearmint) leaf oil, Citrus Aurantifolia (lime) oil, Melaleuca Alternifolia (tea tree) leaf oil, Citrus Grandis (grapefruit) peel oil, Citrus Medica Limonum (lemon) peel oil, rose flower oil, eucalyptus globulus leaf oil, and combinations thereof.
(ii) synthetic ethers containing from 10 to 40 carbon atoms;
(iii) linear or branched hydrocarbons of mineral or synthetic origin, such as petroleum jelly, polydecenes, hydrogenated polyisobutene such as Parleam, and 40 squalane;
(iv) synthetic esters, for instance oils of formula RCOOR′ in which R represents a linear or branched fatty acid residue containing from 1 to 40 carbon atoms and R′ represents a hydrocarbon-based chain that is especially branched, containing from 1 to 40 carbon atoms on condition that R+R′ is ÿ 10, for instance Purcellin oil (cetearyl octanoate), isopropyl myristate, isopropyl palmitate, C12-C15 alkyl benzoate, such as the product sold under the trade name Finsolv TN™ or Witconol TN™ by Witco or Tegosoft TN™ by Evonik Goldschmidt, 2-ethylphenyl benzoate, such as the commercial product sold under the name X-Tend 226 by ISP, isopropyl lanolate, hexyl laurate, diisopropyl adipate, isononyl isononanoate, oleyl erucate, 2-ethylhexyl palmitate, isostearyl isostearate, diisopropyl sebacate, such as the product sold under the name of “Dub Dis” by Stearinerie Dubois, octanoates, decanoates or ricinoleates of alcohols or polyalcohols, such as propylene glycol dioctanoate; hydroxylated esters, such as isostearyl lactate or diisostearyl malate; and pentaerythritol esters; citrates or tartrates, such as di(linear C12-C13 alkyl) tartrates, such as those sold under the name Cosmacol ETI™ by Enichem Augusta Industriale, and also di(linear C14-C15 alkyl) tartrates, such as those sold under the name Cosmacol ETL™ by the same company; or acetates;
(v) fatty alcohols that are liquid at room temperature, containing a branched and/or unsaturated carbon-based chain containing from 12 to 26 carbon atoms, for instance octyldodecanol, isostearyl alcohol, oleyl alcohol, 2-hexyldecanol, 2-butyloctanol or 2-undecylpentadecanol;
(vi) higher fatty acids, such as oleic acid, linoleic acid or linolenic acid;
(vii) carbonates, such as dicaprylyl carbonate, such as the product sold under the name Cetiol CC™ by Cognis;
(viii) fatty amides, such as isopropyl N-lauroyl sarcosinate, such as the product sold under the trade name Eldew SL 205™ from Ajinomoto; and
(ix) essential oils selected from the group consisting of sunflower oil, sesame oil, peppermint oil, macadamia nut oil, tea tree oil, evening primrose oil, sage oil, rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, rosemary oil, sandalwood oil, spearmint oil, spikenard oil, vetiver oil, wintergreen oil, and ylang ylang.

In some embodiments, the the micellar aqueous cleansing composition may include at least one branched or linear liquid alkane with carbon chain length of C11 to C20. In various embodiments, liquid alkanes may be selected from those with a carbon chain length of from C11 to C20. The liquid alkanes may be selected from those with a carbon chain length of from C11 to C20, or from C15 to C19, or one of C11, C12, C13, C14, C15, C16, C17, C18 to C19. In some particular embodiments, suitable liquid alkanes that may be used according to the disclosure include hydrocarbon-based oils containing from 8 to 16 carbon atoms, and especially branched C8-C16 alkanes such as C8-C16 isoalkanes. In some exemplary embodiments, such liquid alkanes may be chosen from isoparaffins, for instance isododecane (also known as 2,2,4,4,6-pentamethylheptane), isodecane, and isohexadecane.

The amount of each of the at least one branched or linear liquid alkane may be present in the micellar aqueous cleansing composition in a range of from about 1% to about 35% by weight, or from about 15% to about 30% by weight, or from about 10% to about 20% or any suitable combination, sub-combination, range, or sub-range thereof by weight, based on the weight of the micellar aqueous cleansing composition. One of ordinary skill in the art, however, will appreciate that other ranges are within the scope of the invention.

Thus, each of the at least one branched or linear liquid alkane in the micellar aqueous cleansing composition may be present by weight, based on the total weight of the micellar aqueous cleansing composition, from about 1, 2, 3, 4, 5, 6, 7, 8, 9, 10, 11, 12, 13, 14, 15, 16, 17, 128, 19, 20, 21, 22, 23, 24, 25, 26, 27, 28, 29, 30, 31, 32, 33, 34, to about 35 percent, including increments and ranges therein and there between.

### Preservatives

In some embodiments, the composition may include as additional ingredients one or more preservatives and/or antimicrobials may be present in the micellar aqueous cleansing composition.

Any preservative commonly used in micellar aqueous cleansing compositions is an acceptable preservative for the micellar aqueous cleansing compositions herein, such as phenoxyethanol, members from the paraben family such as the methyl, ethyl, propyl, butyl or isobutyl parabens, 4-hydroxy benzoic acid, benzoic acid, sorbic acid, dehydroacetic acid, triclosan, benzyl alcohol, chlorophenesin, or salicylic acid, for example. At more concentrated amounts of suitable solvents for optional additives, in particular, suitable solvents for antimicrobials and preservatives, members from the paraben family may be used as a preservative.

In some embodiments, the one or more preservatives, when present, may be selected from phenoxyethanol, caprylyl glycol, Myrtrimonium bromide, hydroxyacetophenone, ethylhexyl glycerin, chlorphenesin, cetrimonium chloride, hexyl glycerin, octylglycerin, benzylglycerin, 3-heptoyl-2,2-propandiol, and 1,2-hexandiol; polyaminopropyl biguanide, also known as polyhexamethylene biguanide, or PHMB, and combinations thereof. In some embodiments, the the micellar aqueous cleansing composition is free or essentially free of one or more of caprylyl glycol, phenoxyethanol, hexyl glycerin, ethylhexylglycerin, octylglycerin, benzylglycerin, 3-heptoyl-2,2-propandiol, and 1,2-hexandiol; polyaminopropyl biguanide, also known as polyhexamethylene biguanide, or PHMB.

In some embodiments, the preservative may comprise one or more of preservatives selected from the group consisting of organic acids, parabens, formaldehyde donors, phenol derivatives, quaternary ammoniums, alcohols, isothiazolinones, and combinations thereof. Preservatives having antibacterial activity are optionally present in the micellar aqueous cleansing compositions of the present invention. Examples of organic acid preservatives include, but are not limited to, sodium benzoate, potassium sorbate, benzoic acid and dehydroacetic acid, sorbic acid, and combinations thereof. A preferred organic acid preservative system includes a mixture of sodium benzoate and potassium sorbate. Examples of paraben preservatives include, but are not limited to, alkyl para-hydroxybenzoates, wherein the alkyl radical has from 1, 2, 3, 4, 5 or 6 carbon atoms and for example, from 1 to 4 carbon atoms e.g., methyl para-hydroxybenzoate (methylparaben), ethyl para-hydroxybenzoate (ethylparaben), propyl para-hydroxybenzoate (propylparaben), butyl para-hydroxybenzoate (butylparaben) and isobutyl para-hydroxybenzoate (isobutylparaben). Examples of formaldehyde donor preservatives include, but are not limited to, 1,3-Dimethylol-5,5-dimethylhydantoin (DMDM hydantoin), imidazolidinyl urea, gluteraldehyde, and combinations thereof. Examples of quaternary ammonium preservatives include, but are not limited to, benzalkonium chloride, methene ammonium chloride, benzethonium chloride, and combinations thereof. Examples of alcohol preservatives include, but are not limited to, ethanol, benzyl alcohol, dichlorobenzyl alcohol, phenoxyethanol, and combinations thereof. Examples of isothiazolone preservatives include, but are not limited to, methylchloroisothiazolinone, methylisothiazolinone, and combinations thereof.

In some embodiments, the preservative includes one or more preservatives, the one or combination present at a concentration, by weight of about 0.001% to about 5%, or alternatively about 0.05% to about 2.5% or alternatively about 0.1% to about 2.0%, or from about 0.02% to about 1% by weight, or from about 0.03% to about 0.5%, or any suitable combination, sub-combination, range, or sub-range thereof by weight, based upon weight of the micellar aqueous cleansing composition. One of ordinary skill in the art, however, will appreciate that other ranges are within the scope of the invention.

Thus, any one of or a combination of preservatives, when present, may be present, by weight, based on the total weight of the micellar aqueous cleansing composition, is from about 0.001, 0.002, 0.003,0.004, 0.005, 0.006, 0.007, 0.008, 0.009, 0.01, 0.02, 0.03,0.04, 0.05, 0.06, 0.07, 0.08, 0.09, 1.0, 1.1, 1.2, 1.3, 1.4, 1.5, 1.6, 1.7, 1.8, 1.9, 2, 3, 4, to about 5 weight percent, including increments and ranges therein and there between.

Although the optional additional ingredients may be given as examples, it will be appreciated that other optional components compatible with cosmetic applications known in the art may be used that are suitable. It will be appreciated by a skilled artisan that any optional additives are present only to the extent and in amounts that do not materially adversely affect the basic and novel characteristic(s) of the claimed disclosure. Thus, In some embodiments, that include optional additives, such optional additives will not materially adversely affect the micellar aqueous cleansing composition.

More generally, it will be appreciated by a skilled artisan that any solvents, humectants, preservatives or other additives are present only to the extent and in amounts that do not materially adversely affect the basic and novel characteristic(s) of the claimed invention.

Although the aforementioned optional components are given as an example, it will be appreciated that other optional components compatible with cosmetic applications known in the art may be used.

Further, any one or more (or all) of additional or optional additives and actives may be excluded from the micellar aqueous cleansing compositions. In other words, the micellar aqueous cleansing compositions may be free or essentially free of any or all of the additional or optional additives, actives, and other ingredients disclosed herein.

In accordance with the various embodiments, the amount of one or more actives and additives, alone or in combination, when present in the micellar aqueous cleansing composition according to the disclosure can be present in a range from about 0.0001% to about 20%, and In some embodiments, from about 0.005% to about 0.01%, and In some embodiments, from about 0.01% to about 0.1%, and In some embodiments, from about 0.15% to about 5%, and In some embodiments, from about 0.40% to about 4%, and In some embodiments, from about 0.5% to about 2.5%, and In some embodiments, from about 0.1% to about 0.5% and In some embodiments, from about 1% to about 2%, or any suitable combination, sub-combination, range, or sub-range thereof by weight, based on the total weight of the micellar aqueous cleansing composition. One of ordinary skill in the art, however, will appreciate that other ranges are within the scope of the invention.

Thus, any one or a combination of actives and additives may be present, each one or the combination present from about 0.0001, 0.0005, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1.0, 2, 3, 4, 5, 6, 7, 8, 9, 10, 11, 12, 13, 14, 15, 16, 17, 18, 19 to about 20 percent, by weight, including increments and ranges therein and there between.

### Examples

### Example 1: Inventive Compositions

Various representative embodiments of the inventive and comparative compositions are exemplified herein.

TABLE 1
Inventive Compositions

| INCI | Inventive 1 | Inventive 2 | Inventive 3 | Inventive 4 | Inventive 5 |
|---|---|---|---|---|---|
| sodium methyl | 4.8 | 4.8 | 4.8 | 4.8 | 4.8 |
| cocoyl taurate | | | | | |
| cocamidopropyl | 7.5 | 7.5 | 7.5 | 7.5 | 7.5 |
| hydroxysultaine | | | | | |
| lauryl lactate | 0.65 | 0.65 | 0.65 | 0.65 | 0.65 |
| polyquaternium-47 | 0.105 | 0.5 | | | |
| polyquaternium-22 | | | 0.5 | | |
| polyquaternium-39 | | | | 0.5 | |
| polyquaternium-67 | | | | | 0.5 |
| hexylene glycol | 0.5 | 0.5 | 0.5 | 0.5 | 0.5 |
| sodium hydroxide | 0.4 | 0.4 | 0.4 | 0.4 | 0.4 |
| menthol | 0.1 | 0.1 | 0.1 | 0.1 | 0.1 |
| zinc gluconate | 0.2 | 0.2 | 0.2 | 0.2 | 0.2 |
| tetrasodium | 0.0475 | 0.0475 | 0.0475 | 0.0475 | 0.0475 |
| glutamate diacetate | | | | | |
| sodium benzoate | 0.001 | 0.001 | 0.001 | 0.001 | 0.001 |
| glycerin | 5.0 | 5.0 | 5.0 | 5.0 | 5.0 |
| sodium chloride | 2.12 | 2.12 | 2.12 | 2.0 | 2.0 |
| capryloyl salicylic | 0.05 | 0.05 | 0.05 | 0.05 | 0.05 |
| acid | | | | | |
| salicylic acid | 2.0 | | | | |
| water | QS/~78 | QS/~78 | QS/~79 | QS/~79 | QS/~79 |
| STABILITY | Stable 2M | Stable 2M | Stable 2M | Stable 2M | Stable 2M |

### Example 2: Benchmark Composition

The commercial benchmark includes PEG/Sulfate and lacks lauryl lactate: hexylene glycol (0.50%), PEG-6 caprylic/capric glycerides (0.32%), PEG-150 pentaerythrityl tetrastearate (0.80%), sodium hydroxide (0.47%), polyquaternium-47 (0.105%), menthol (0.10%), zinc gluconate (0.20%), tetrasodium glutamate diacetate (%), sodium benzoate (0.001%), glycerin (3.00%), citric acid (0.00%), sodium methyl cocoyl taurate (%), lauryl lactate (%), tetrasodium EDTA (0.17%), cocamidopropyl hydroxysultaine (%), coco-betaine (2.10%), decyl glucoside (3.18%), sodium laureth sulfate (10.15%), salicylic acid (2.00%), sodium chloride (2.955%), water (76.399%), and capryloyl salicylic acid (0.05%).

### Example 2: Comparative Compositions

TABLE 2
Comparative Compositions (Inventives without Lauryl Lactate)

| Comparative 1 | Comparative 2 | Comparative 3 | Comparative 4 |
|---|---|---|---|
| (INV 1 w/o Lauryl | (INV 3 w/o Lauryl | (INV 4 w/o Lauryl | (INV 5 w/o Lauryl |
| Lactate) | Lactate) | Lactate) | Lactate) |
| Unstable after 2 | Unstable AFTER 3 | Unstable AFTER 2 | Unstable AFTER 2 |
| weeks | weeks | weeks | week s |
| Phase Separation | Phase Separation | Phase Separation | Phase Separation |

TABLE 3
Comparative Compositions (Inventives with substitutions for Lauryl Lactate)

| Comparative 2A (INV 3 w | Comparative 2B (INV 3 w | |
|---|---|---|
| xanthan gum, no lauryl | Hydroxyethylcellulose, no | Comparative 2C (INV 3 w |
| lactate) | lauryl lactate) | algin, no lauryl lactate) |
| 45° C. 1 month unstable | 45° C. 1 month unstable | 45° C. 1 month unstable |
| Phase Separation | Phase Separation | Phase Separation |

### Example 3: Foaming Tests

Foaming of compositions was evaluated using the following Instrument: Kruss DFA100 Dynamic Foam Analyzer, and the results are shown in Table 4, below.

For foaming analysis, a Dynamic foam analyzer (Kruss DFA100) was used. Each evaluated composition was diluted at 25× to evaluate foaming performance. Foam was generated via mechanical stirring in first 20 seconds and foam stability was monitored in following 180 seconds. The results are shown in Table 4, below.

Comparatives that are unstable, as shown above, were not tested for foaming due to instability of structure of formulation to be properly evaluated for foaming.

TABLE 4
Foaming Analysis

| | Inventive 1 | Inventive 2 | Inventive 3 | Inventive 4 | Inventive 5 |
|---|---|---|---|---|---|
| Foam High (mL) | 220.3 | 235.6 | 252.6 | 248.6 | 231.5 |
| Testing on Face | Middle - low | Middle | Middle - high | Middle - high | Middle |
| Foaming (In- | | | | | |
| Vivo) | | | | | |

### Example 4: Viscosity

Viscosity analysis results are shown below in Table 5 according to the method described herein above. Comparatives that are unstable, as shown above, were not tested for viscosity due to instability of structure of formulation (indicated as ND).

TABLE 5
Viscosity analysis

| | Inv 2 | Comp 1 | Inv 3 | Comp 2 | Inv 4 | Comp 3 | Inv 5 | Comp 4 |
|---|---|---|---|---|---|---|---|---|
| Viscosity | 35.2 | <0 (very | 28.3 | 4.2 | 25.6 | <0 (very | 24.6 | <0 (very |
| (M4) UD | | liquid) | | | | liquid) | | liquid) |
| 25° C. | | | | | | | | |
| Viscosity | 33.6 | <0 (very | 26.5 | ND | 23.8 | ND | 23.4 | ND |
| (M4) UD | | liquid) | | | | | | |
| 45° C. | | | | | | | | |

While the disclosure has been described with reference to an embodiment, it will be understood by those skilled in the art that various changes may be made and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure not be limited to the particular embodiment disclosed as the best mode contemplated for carrying out this disclosure, but that the disclosure will include all embodiments falling within the scope of the appended claims.

The articles “a” and “an,” as used herein, mean one or more when applied to any feature in embodiments of the present disclosure described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used. The adjective “any” means one, some, or all indiscriminately of whatever quantity.

“At least one,” as used herein, means one or more and thus includes individual components as well as mixtures/combinations.

The transitional terms “comprising,” “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinarily associated with the specified material(s). The term “consisting essentially of” limits the scope of a claim to the specified elements, steps or material(s) and those that do not materially affect the basic and novel characteristic(s) of the claimed disclosure. All materials and methods described herein that embody the present disclosure can, in alternate embodiments, be more specifically defined by any of the transitional terms “comprising,” “consisting essentially of,” and “consisting of.”

“Free” and “devoid” and “exclude” or “excluded” each indicates that no reliably measurable excluded material, for example, an excluded thickener or excluded sulfate surfactant or other excluded material as described herein, is present in the micellar aqueous cleansing composition. The term “essentially free” means that, while it is preferred that no excluded material is present in the micellar aqueous cleansing composition, it is possible to have very small amounts of the excluded material in the micellar aqueous cleansing composition of the invention, provided that these amounts do not materially affect the advantageous properties of the micellar aqueous cleansing composition. In particular, “essentially free” means that excluded material can be present in the micellar aqueous cleansing composition at an amount of less than 5%, or less than 4%, or less than 3%, or less than 2%, or less than 1%, or less than 0.1% by weight, or 0%, based on the total weight of the micellar aqueous cleansing composition.

In accordance with the various embodiments, it is contemplated that some or all of following ingredients may be excluded from the micellar aqueous cleansing composition according to the invention: alcohols, for example but not limited to, monohydric alcohols; powders comprising Nylon-12, polymethyl methacrylate, polyethylene beads, and powders which are considered microplastics; lactic acid, glycolic acid, tartaric acid, mandelic acid, citric acid, salicylic acid and derivatives thereof (including 5-n-octanoylsalicylic acid, salicylate, sodium salicylate, and willow extract), salicylic acid, capryloyl salicylic acid, beta hydroxybutanoic acid, propionic acid, beta-hydroxy beata-methylbutyric acid, carnitine tropic acid, and trethocanic acid; alkyl polyglucosides selected from the group consisting of decyl glucoside, lauryl glucoside, octyl glucoside, coco glucoside, caprylyl/capryl glucoside, and sodium lauryl glucose carboxylate, and combinations thereof; surfactants with a C10-C20 fatty alcohol or acid hydrophobe condensed with from about 2 to about 100 moles of ethylene oxide or propylene oxide per mole of hydrophobe; C2-C10 alkyl phenols condensed with from 2 to 20 moles of alkylene oxide; mono- and di-fatty acid esters of ethylene glycol; fatty acid monoglyceride; sorbitan; mono- and di-C8-C20 fatty acids; polyoxyethylene sorbitan; alkyl polyglycosides and saccharide fatty amides (e.g. methyl gluconamides); alkyl ether sulfate and sulfonates; alkyl sulfates and sulfonates; alkylbenzene sulfonates; alkyl and dialkyl sulfosuccinates; C8-C20 acyl isethionates; C8-C20 alkyl ether phosphates; alkylethercarboxylate, PEG-100 Stearate; PEG-20 Stearate and other esters of Poly(Ethylene Glycol); polyethylene glycol (PEG) surfactants, polypropylene glycol (PPG) surfactants, sulfate surfactants; taurates, for example, taurate surfactants; Sucrose Stearate and other emulsifiers based on sugar esters; Glyceryl Stearate and other glycerol esters; Disodium Ethylene Dicocamide PEG-15 Disulfate; Sodium Steroyl Glutamate and other fatty amides; Steareth-100 and other fatty ethers, cationic surfactants, and other cationic compounds; silicones, such as silicone polymers, silicone elastomers, and silicone oils, for example selected from dimethicone, cyclopentasiloxane, cyclohexasiloxane, and combinations thereof; phthalates, parabens, sulfates, polyquaternium, microplastics, synthetic dyes, gelling agents, and combinations thereof.

“Excluded polymeric thickeners” means and refers to thickeners that have not been added as a component in the micellar aqueous cleansing composition or that may be excluded in some embodiments. Thus, in some embodiments, the micellar aqueous cleansing composition is essentially free, free or devoid of one or more of PEG/PEO polymeric thickeners, nature based thickeners, and synthetic thickeners. In some embodiments, the micellar aqueous cleansing composition is essentially free of or excludes PEG-type polymeric thickeners selected from PEG-150 pentaerythrityl tetrastearate, PEG-120 thickeners, PEG-150 thickeners, PEG-80 glyceryl tallow ester, PEG-8 PPG (polypropylene glycol)-3 diisostearate, PEG-200 hydrogenated glyceryl palmitate, PEG-n(n=6, 8, 12) beeswax, PEG-4 isostearate, PEG-n(n=3, 4, 8, 150) distearate PEG-n(n=28, 200) glyceryl tallow ester, PEG-7 hydrogenated castor oil, PEG-40 jojoba oil, PEG-120 methyl glucose dioleate, PEG-150 pentaerythritol stearate, PEG-55 propylene glycol oleate, PEG-160 sorbitan triisostearate, SMDI copolymer (polyethylene glycol-150/decyl/methacrylate copolymer), PEG-150/stearyl/SMDI copolymer, PEG-90, and combinations thereof. In some embodiments, the micellar aqueous cleansing composition may exclude one or more polymers such as cationic polymers selected from polylysine, chitosan, and guar. In some embodiments, the micellar aqueous cleansing composition may exclude one or more polymers selected from polysaccharides that include alginates/algin, chitosan, cyclodextrin, cationic gelatin, cationic dextran, celluloses, cationic cellulose, chitin, starches, galactomannans such as guar gums and its derivatives, including hydroxypropyl guar, cationic guar derivatives, gums of microbial origin, including xanthan gum, scleroglucan gum, mucopolysaccharides, chondroitin sulfates, and mixtures thereof, and gums derived from plant exudates, including locust bean gums, gum arabic, ghatti gum, karaya gum, gum tragacanth, carrageenan gum, agar gum and carob gum, cellulose-based polymers, including methylcellulose, hydroxyalkylcellulose, ethylhydroxyethylcelluloses, hydroxyethylcellulose, hydroxypropylcellulose, carboxymethylcellulose, cationic cellulose ether derivatives, quaternized cellulose derivatives, nitrocellulose, hemicellulose and hemicellulose derivatives, mannans, xylans, lignins, arabans, galacturonans, alginate-based compounds, glucuronoxylans, arabinoxylans, xyloglucans, glucomannans, fructosans such as inulin, pectic acids and pectins, arabinogalactans, agars, glycosaminoglycans, gelatin, gellan, crosslinked homopolymers of acrylic acid, cationic peptides and their derivatives (e.g., polylysine, polyornithine), peptide/protein polymers (e.g., histone, collagen); cationic thiolated biopolymers (nature-based thiomers or nature-based dendrimers, e.g., chitosan-cysteine, chitosan-thiobutylamidine, chitosan-thioglycolic acid); algin, xanthan gum (and) sclerotium gum (and) lecithin (and) pullulan (Siligel™), xanthan gum, carbomer 980, hydroxypropyl starch phosphate, ammonium polyacryloyldimethyl taurate, hydroxyethyl cellulose, acrylate copolymer, sclerotium gum; or combinations thereof. In some embodiments, the composition may also exclude one or more PEG type compound selected from poloxamer 184 (polyoxyethylene polyoxypropylene glycol), PEG-20 glyceryl triisostearate, PEG-7 glyceryl cocoate, PEG-20 methylglucoside sesquistearate, PG-5 dioleate, PG-4 diisostearate, PG-10 isostearate, PEG-60 hydrogenated castor oil, PEG-8 stearate, PEG-8 isostearate, PEG-60 hydrogenated castor oil, PEG-150 Laurate, PEG-150 Distearate, PEG-150 pentaerythrityl tetrastearate, PEG-78 Glyceryl Cocoate, PEG-30 Glyceryl Cocoate, PEG-type polymeric thickeners selected from PEG-150 pentaerythrityl tetrastearate, PEG-120 thickeners, PEG-150 thickeners, PEG-80 glyceryl tallow ester, PEG-8 PPG (polypropylene glycol)-3 diisostearate, PEG-200 hydrogenated glyceryl palmitate, PEG-n(n=6, 8, 12) beeswax, PEG-4 isostearate, PEG-n(n=3, 4, 8, 150) distearate PEG-n(n=28, 200) glyceryl tallow ester, PEG-7 hydrogenated castor oil, PEG-40 jojoba oil, PEG-120 methyl glucose dioleate, PEG-150 pentaerythritol stearate, PEG-55 propylene glycol oleate, PEG-160 sorbitan triisostearate, SMDI copolymer (polyethylene glycol-150/decyl/methacrylate copolymer), PEG-150/stearyl/SMDI copolymer, PEG-90, -20, Ceteth-10, Ceteth-20, Isoceteth-20, Laureth-4, Laureth-23, Oleth-10, Oleth-20, Steareth-10, Steareth-20, Steareth-100, and Steareth-21, PEG-20 glyceryl laurate, PEG-40 hydrogenated castor oil, or combinations thereof.

“Sulfate surfactant-free” means that excluded surfactants have not been added as a component. In some particular embodiments, the micellar aqueous cleansing composition is devoid of sulfate based surfactant. In some embodiments, the micellar aqueous cleansing composition is devoid of ethylene glycol (PEG) based surfactants. Those of skill in the art will appreciate that a surfactant may be present in a composition via its presence in one or more of the formulation components; thus, in some embodiments the micellar aqueous cleansing composition may be “essentially-free” wherein the excluded surfactant is present at a concentration that does not exceed 5% by weight, and in some instances is present not more than 3% by weight, and in some instances is present not more than 1% by weight, based on the weight of the cosmetic micellar aqueous cleansing composition. In some particular embodiments “sulfate surfactant-free” means that the cosmetic micellar aqueous cleansing composition is free or devoid specifically of the excluded sulfate surfactant.

“Petrochemical-free” means that excluded petrochemical have not been added as a component. Some specific but non-limiting examples of petrochemicals that are lacking from the cosmetic micellar aqueous cleansing composition include benzalkonium chloride, isododecane, isohexadecane and the like. In some embodiments, the micellar aqueous cleansing composition is not free from petrochemicals, but is formulated to have an amount of petrochemicals that is at or below the amounts found in similar compositions that are commercially available. The micellar aqueous cleansing composition may be petrochemical-free.

“Silicone-free” means that excluded silicones have not been added as a component. In some embodiments, a composition is devoid of silicones. Some specific but non-limiting examples of silicones that are lacking from the cosmetic micellar aqueous cleansing composition includes, but is not limited to, silicone polymers, for example selected from dimethicone, cyclopentasiloxane, and other silicone oils, and silicone elastomers. The micellar aqueous cleansing composition may be silicone-free.

Other than in the operating examples, or where otherwise indicated, all numbers expressing quantities of ingredients and/or reaction conditions are to be understood as being modified in all instances by the term “about,” meaning within 10% of the indicated number (e.g., “about 10%” means 9%-11% and “about 2%” means 1.8%-2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used, and may or may not reflect the final percentage of the active, wherein the final percentage of the active is dependent on the weight percent of active in the raw material.

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “²% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements. The example that follows serves to illustrate embodiments of the present disclosure without, however, being limiting in nature.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

What is claimed is:

1. A micellar aqueous cleansing composition comprising:
a) at least one natural thickener comprising lauryl lactate; and
b) at least one or a combination of polyquaternium cationic polymers;
wherein the micellar aqueous cleansing composition excludes any added PEG-based ingredients, including PEG/PEO polymeric thickeners, and excludes sulfate-based ingredients, including sulfate-based surfactants, and
wherein the micellar aqueous cleansing composition has a gel-like viscosity and is transparent in appearance.

2. The micellar aqueous cleansing composition according to claim 1, wherein lauryl lactate is present from about 0.05% to about 2%, by weight based on the weight of the micellar aqueous cleansing composition.

3. The micellar aqueous cleansing composition according to claim 1, wherein lauryl lactate is present from about 0.1% to about 1%, by weight, based on the weight of the micellar aqueous cleansing composition.

4. The micellar aqueous cleansing composition according to claim 1, wherein the at least one natural thickener consists essentially of lauryl lactate and excludes any added natural thickener.

5. The micellar aqueous cleansing composition according to claim 1, wherein the composition excludes other natural thickeners such as xanthan gum, sclerotium gum, alginate, hydroxyethylcellulose, Zea Mays starch, carrageenan

6. The micellar aqueous cleansing composition according to claim 1, wherein the at least one or combination of polyquaternium cationic polymers is selected from the group consisting of polyquaternium-22, 39, 47, 67, and combinations thereof.

7. The micellar aqueous cleansing composition according to claim 1, wherein the polyquaternium cationic polymers consist of one of polyquaternium-22, 39, 47, or 67.

8. The micellar aqueous cleansing composition according to claim 1, wherein each one of the polyquaternium cationic polymers is present from about at least 0.5%, by weight, based on the weight of the micellar aqueous cleansing composition.

9. The micellar aqueous cleansing composition according to claim 1, wherein each one of the polyquaternium cationic polymers is present from about 0.05% to about 1%, by weight, based on the weight of the micellar aqueous cleansing composition.

10. The micellar aqueous cleansing composition according to claim 1, wherein the composition demonstrates stability including remaining visibly clear, maintaining viscosity, micellar phase stability free of separation, all at ambient temperatures and at high temperatures.

11. The micellar aqueous cleansing composition according to claim 1, comprising one or more surfactants.

12. The micellar aqueous cleansing composition according to claim 11, wherein the composition comprises one or more amphoteric surfactants comprising at least hydroxysultaine and one or more anionic surfactants comprising sodium methyl cocoyl taurate.

13. The micellar aqueous cleansing composition according to claim 12, wherein cocoamidopropyl hydroxysultaine is present in a range from about 1% to about 20%, by weight, based on the weight of the micellar aqueous cleansing composition.

14. The micellar aqueous cleansing composition according to claim 12, wherein sodium methyl cocoyl taurate is present from about 0.1% to about 1.5%, by weight, based on the weight of the micellar aqueous cleansing composition.

15. The micellar aqueous cleansing composition according to claim 1, wherein water is present in a range from about 40% to about 90%, by weight, based on the weight of the composition.

16. The micellar aqueous cleansing composition according to claim 1, comprising one or more ingredients selected from the group consisting of hexylene glycol, sodium hydroxide, menthol, zinc gluconate, tetrasodium glutamate diacetate, sodium benzoate, glycerin, sodium chloride, salicylic acid, capryloyl salicylic acid, and combinations thereof.

17. The micellar aqueous cleansing composition according to claim 16, comprising each of hexylene glycol, sodium hydroxide, menthol, zinc gluconate, tetrasodium glutamate diacetate, sodium benzoate, glycerin, sodium chloride, salicylic acid, capryloyl salicylic acid, and water.

18. The micellar aqueous cleansing composition according to claim 1, wherein the composition excludes PEG-150 pentaerythrityl tetrastearate, PEG-6 caprylic/capric glycerides, or a combination thereof.

19. A micellar aqueous cleansing composition comprising:
a) at least one natural thickener comprising lauryl lactate, wherein lauryl lactate is present from about 0.1% to about 1%; and
b) at least one or a combination of polyquaternium cationic polymers selected from the group consisting of polyquaternium-22, 39, 47, 67, and combinations thereof, wherein each of the polyquaternium cationic polymers is present from about at least 0.5%;
all amounts by weight, based on the weight of the micellar aqueous cleansing composition,
wherein the micellar aqueous cleansing composition excludes any added PEG-based ingredients, including PEG/PEO polymeric thickeners, and excludes sulfate-based ingredients, including sulfate-based surfactants, and
wherein the micellar aqueous cleansing composition demonstrates stability including remaining visibly clear, maintaining viscosity, micellar phase stability free of separation, all at ambient temperatures and at high temperatures, has a gel-like viscosity, and is transparent in appearance.

20. A micellar aqueous cleansing composition comprising:
a) at least one natural thickener consisting essentially of lauryl lactate, wherein lauryl lactate is present from about 0.1% to about 1%;
b) polyquaternium cationic polymers consisting of one of polyquaternium-22, 39, 47, or 67, wherein each of the polyquaternium cationic polymers is present from about at least 0.5%;
c) one or more amphoteric surfactants comprising at least hydroxysultaine present in a range from about 1% to about 20% and one or more anionic surfactants comprising sodium methyl cocoyl taurate present from about 0.1% to about 1.5%; and
d) one or more ingredients selected from the group consisting of hexylene glycol, sodium hydroxide, menthol, zinc gluconate, tetrasodium glutamate diacetate, sodium benzoate, glycerin, sodium chloride, salicylic acid, capryloyl salicylic acid, and combinations thereof,
all amounts by weight, based on the weight of the micellar aqueous cleansing composition,
wherein the micellar aqueous cleansing composition excludes PEG-150 pentaerythrityl tetrastearate, PEG-6 caprylic/capric glycerides, or a combination thereof, and
wherein the micellar aqueous cleansing composition has a gel-like viscosity and is transparent in appearance.
